(19) United States
(12) Patent Application Publication
(10) Pub. No.: US 2014/0066606 A1
(43) Pub. Date: Mar. 6, 2014

(54) PRODUCTION OF HIGH MANNOSE GLYCOSYLATED PROTEINS STORED IN THE PLASTID OF MICROALGAE

(71) Applicants: Aude Carlier, Nantes (FR); Remy Michel, Nantes (FR); Jean-Paul Cadoret, Basse Goulaine (FR); Alexandre Lejeune, La Chapelle Sur Erdre (FR); Nathalie Dufourmantel, Fay de Bretagne (FR)

(72) Inventors: Aude Carlier, Nantes (FR); Remy Michel, Nantes (FR); Jean-Paul Cadoret, Basse Goulaine (FR); Alexandre Lejeune, La Chapelle Sur Erdre (FR); Nathalie Dufourmantel, Fay de Bretagne (FR)

(73) Assignee: ALGENICS, Saint Herblain (FR)

(21) Appl. No.: 13/997,670

(22) PCT Filed: Dec. 28, 2011
(86) PCT No.: PCT/EP2011/006592

§ 371 Date: Jun. 25, 2013

(30) Foreign Application Priority Data
Dec. 29, 2010 (EP) 10016162.9

(51) Int. Cl. C12N 15/82; C07K 14/505; C12Q 1/34; C07K 14/005; C12P 21/00; G01N 33/68

(52) U.S. Cl. CPC C12N 15/8258; C12P 21/005; G01N 33/6842; C12Q 1/34; C07K 14/005; C07K 14/505
USPC 530/395; 435/257.2; 435/69.8; 435/7.92; 435/18

(57) ABSTRACT

The present invention concerns a transformed microalga producing a protein harboring a “high mannose” pattern of glycosylation in the plastid of the transformed microalga, wherein 1) the transformed microalga has a Chloroplast Endoplasmic Reticulum (CER); 2) the microalga has been transformed with a nucleic acid sequence operatively linked to a promoter, the nucleic acid sequence encoding an amino acid sequence including (i) an amino-terminal bipartite topogenic signal (BTS) sequence composed of at least a signal peptide followed by a transit peptide; and (ii) The sequence of the protein, 3) the xylosyltransferases and fucosyltransferases of the microalga have not been inactivated; 4) the N-acetylglycosyltransferase I of the microalga has not been inactivated, preferably the N-acetylglycosyltranferases II, III, IV, V and VI, mannosidase II and glycosyltransferases of the microalga have not been inactivated.

This patent application claims the priority of European patent application EP10016162.9 filed on Dec. 29, 2010, which is incorporated herein by reference.

### FIELD OF THE INVENTION

The present invention is directed to the use of a transformed microalga for the production of glycosylated proteins harboring a pattern of N-glycosylation with high mannose residues, said glycosylated proteins being targeted and stored in the plastid of said microalga. This invention also encompasses methods of producing said glycosylated proteins.

### BACKGROUND OF THE INVENTION

The increasing demand for recombinant drugs has strongly driven the development of various cellular models used as biomanufacturing platforms. Expression systems based on eukaryotic cells are preferentially used for the production of recombinant proteins requiring post-translational modifications (PTM) for their biological activity and/or stability. N-glycosylation is a major PTM characterized by the attachment of glycans onto certain asparagine residues of proteins. N-glycosylation starts when the protein is co-translationally imported into the Endoplasmic Reticulum (ER) leading to mannose-type N-glycans having from 9 to 5 mannose residues which are highly conserved in eukaryotes. Further processing of N-glycans occurs in the Golgi apparatus through the action of various glycosyltransferases. This step leads to mature N-glycosylated proteins harboring organism-specific complex N-glycan structures. Moreover, for a given glycoprotein, N-glycans are often not homogeneous resulting in a pool of structurally distinct oligosaccharides and therefore various glycoforms of said glycoprotein. Complex glycans and heterogeneity can constitute drawbacks of currently available expression systems for the production of recombinant glycoproteins. The occurrence of various glycoforms can thus compromise batch-to-batch consistency resulting in quality and regulatory issues in the production of recombinant drugs. Organism-specific complex N-glycans can also result in issues when using non-human cells. For example, glycoproteins produced in CHO cells can contain N-glycolylneuraminic acid whereas those produced in murine cells contain galactose-α(1,3)-galactose which are respectively potentially and highly immunogenic for human. Similarly, insect- or plant-based expression systems also contain potentially immunogenic epitopes such as α(1,3)-fucose (in insect and plant cells) and β(1,2)-xylose (in plant cells).

The present invention discloses the production of glycoproteins and their storage in the plastid of transformed microalgae from the groups of heterokontophytes, haptophytes or cryptophytes. These groups are unique amongst other microalgae or even photosynthetic eukaryotic organisms as they contain plastids surrounded by four membranes with the outermost membrane being interconnected with the ER membrane and studded with ribosomes. This outermost membrane is commonly named Chloroplast Endoplasmic Reticulum (CER). A pathway has been characterized for the targeting of nuclear-encoded proteins in the plastid of these microalgae. Precursors of those proteins are synthesized with an amino-terminal bipartite targeting signal sequence also called “bipartite topogenic signal” (BTS) sequence composed of a signal peptide followed by a transit peptide preceding the sequence of the mature protein. The first step of trafficking in these microorganisms involves the co-translational transport into the ER lumen via the signal peptide. The mechanism for crossing through the second outermost membrane has not been fully identified. Passage through the two innermost membranes to reach the stroma likely involves the transit peptide and translocators (see Bolte et al. (2009) Protein targeting into secondary plastids, Journal of Eukaryotic Microbiology, n^{o} 56, pp: 9-15 for a review of plastids targeting). The evidences of a transport of nuclear-encoded glycosylated proteins to the plastid are scarce and concern plant plastids surrounded by two membranes. These glycoproteins contain complex N-glycan patterns such as β(1,2)-xylose and α(1,3)-fucose residues typical of those added in the Golgi apparatus. The existing literature does not reveal any data concerning the transport of nuclear-encoded glycoproteins to microalgal plastids surrounded by a CER membrane. Besides, background art does not teach or suggest any method for the production of glycoprotein and their storage in the plastidial compartment.

The inventors have surprisingly discovered that CER microalgae can be used as very efficient producing tools for the high yields and stable production of proteins harbouring a homogenous “high mannose” glycosylation pattern, said proteins being afterward easily purified from the plastid of said microalgae.

In fact, the use of a bipartite topogenic signal sequence in a transformed microalga having a CER enabled the expression and glycosylation of proteins in the Endoplasmic Reticulum followed by a transport into the plastid of said microalga without any passage through the Golgi apparatus. N-linked glycans of these glycoproteins are high mannose oligosaccharides (Man-5 to Man-9) characteristic of the ER glycosylation pattern and consequently do not present immunogenic patterns of glycosylation such as those added by glycosyl transferases into the Golgi apparatus. Therefore, the present invention offers an effective method for the production of therapeutic recombinant proteins requiring high mannose glycans for their biological activity without having to inactivate the glycosylation pathway in the Golgi apparatus. Proteins harboring a “high mannose” pattern of glycosylation hold a strong therapeutic interest in the treatment of various diseases. For example, recombinant lysosomal enzymes used for the treatment of lysosomal storage disorders such as Gaucher's or Fabry's diseases require terminal mannose residues for their uptake by human cells. Such glycosylation pattern cannot be directly obtained by CHO cells used for the commercial production of current enzyme replacement therapies and enzymes produced by this system need further deglycosylation steps. The present invention has also applications for the production of viral envelope proteins often considered as difficult-to-express proteins in animal cells. For example, the native glycoprotein gp120 of the HIV envelope spike bears N-linked glycans which are almost entirely oligomannose (Man₅₋₉GlcNAc₂). These glycans of gp120 (Man₆₋₉GlcNAc₂) are important determinant of antibodies recognition including 2G12, one of the most effective HIV neutralizing antibody. In the context of the viral vaccination design, the present invention thus confers a major advantage over conventional platform such as human cell lines for the production of the envelope glycoproteins bearing high mannose glycans, and used as antigens.

The glycans' homogeneity as opposed to mixture of complex glycan structures obtained in other expression systems also constitutes a major benefit of the present invention in term of product quality and consistency. Furthermore, said method is also effective for the production of a high amount of proteins in a stable environment as said proteins are transported and stored into the plastidial stroma.

### SUMMARY OF THE INVENTION

The present invention describes a microalgal-based expression system for the production of glycoproteins and their storage in the plastid. Microalgae used for this invention are species from the groups of heterokonts, cryptophytes and haptophytes that harbor plastid surrounded by an outermost membrane continuous with the Endoplasmic Reticulum. Glycoproteins expressed by mean of the present invention contain targeting sequence allowing their co-translational import into the ER where they undergo N-glycosylation prior to their transport into the plastidial stroma. The present invention enables the production of glycoproteins having a N-glycosylation pattern composed of “high mannose” oligosaccharides, preferably said N-glycosylation pattern being non-immunogenic.

Therefore, a first object of the invention relates to a transformed microalga producing at least one protein harboring a “high mannose” pattern of glycosylation in the plastid of said transformed microalga, wherein
1) said transformed microalga has a Chloroplast Endoplasmic Reticulum (CER);
2) said microalga has been transformed with a nucleic acid sequence operatively linked to a promoter, said nucleic acid sequence encoding an amino acid sequence comprising:
   (i) An amino-terminal bipartite topogenic signal (BTS) sequence composed of at least a signal peptide followed by a transit peptide; and
   (ii) The sequence of said protein;
3) the xylosyltransferases and fucosyltransferases of said microalga have not been inactivated;
4) The N-acetylglycosyltransferase I of said microalga has not been inactivated, preferably the N-acetylglycosyltranferases II, III, IV, V and VI, mannosidase II and glycosyltransferases of said microalga have not been inactivated.

Another object of the invention relates to a method for producing at least one protein harboring a “high mannose” pattern of glycosylation in the plastid of a transformed microalga having a Chloroplastic Endoplasmic Reticulum (CER) as claimed in any one of claims 1 to 10, said method comprising the steps of:
1) Culturing said transformed microalga;
2) Harvesting the plastid of said transformed microalga;
3) Purifying said protein from said plastid.

Still another object of the invention relates to a protein harboring a high mannose pattern of glycosylation produced by the method of the invention.

Another object of the invention provides a pharmaceutical composition comprising a protein according to the invention and eventually a pharmaceutically acceptable carrier.

Finally, another object of the invention relates to the use of a transformed microalga having a CER according to the invention for the production of a polypeptide harboring a “high mannose” pattern of glycosylation in the plastid of said microalga.

### BRIEF DESCRIPTION OF DRAWINGS

FIG. 1. Subcellular localization of platid-targetd EPO-eGFP studied by confocal microscopy. A: bright field; B: chlorophyll autofluorescence shown in red; C: eGFP fluorescence shown in green; D and E: red-green merged images.

FIG. 2. Expression of EPO-eGFP proteins were detected by Immunoblotting with anti-eGFP antibody (A) and anti-EPO antibody (B). wt: wild-type cells; eGFP-EPO (ER): ER-retained eGFP-EPO; eGFP: enhanced Green Fluorescent Protein produced in Escherichia coli; EPO: commercial erythropoietin; Cl 1-3: clones from independent cell lines transformed by a vector carrying plastid-targeted EPO-eGFP (EPO-eGFP plastid).

FIG. 3. Comparative immunoblotting of EPO-eGFP before (nd) and after deglycosylation by Peptide N-Glycosidase F (P) and endoglycosidase H (E) using anti-eGFP antibody (A) and anti-EPO antibody (B). eGFP-EPO (ER): ER-retained eGFP-EPO; EPO-eGFP (plastid): plastid-targeted EPO-eGFP; wt: wild-type cells.

FIG. 4. Expression of gp120-eGFP protein was detected by Immunoblotting with anti-eGFP antibody. wt: wild-type cells; eGFP: enhanced Green Fluorescent Protein produced in Escherichia coli; Cl 1-6: clones from independent cell lines transformed by a vector carrying plastid-targeted gp120-eGFP.

FIG. 5. Comparative immunoblotting of plastid-targeted gp120-eGFP before (nd) and after deglycosylation by Peptide N-Glycosidase F (P) and endoglycosidase H (E) using anti-eGFP antibody. Cl 1-2: clones from independent cell lines transformed by a vector carrying plastid-targeted gp120-eGFP; wt: wild-type cells.

### DETAILED DESCRIPTION OF THE INVENTION

The invention aims to provide a new system for producing proteins harboring a “high mannose” pattern of glycosylation, said proteins being expressed and glycosylated in the Endoplasmic Reticulum of microalgae and further transported in the plastid of said microalgae without any passage through the Golgi apparatus.

An object of the invention is the use of a transformed microalga for the production of at least one protein harboring a “high mannose” pattern of glycosylation in the plastid of said transformed microalga, wherein
1) said transformed microalga has a Chloroplast Endoplasmic Reticulum (CER);
2) said microalga is transformed with a nucleic acid sequence operatively linked to a promoter, said nucleic acid sequence encoding an amino acid sequence comprising:
   (i) An amino-terminal bipartite topogenic signal (BTS) sequence composed of at least a signal peptide followed by a transit peptide; and
   (ii) The sequence of said protein.
3) the xylosyltransferases and fucosyltransferases of said microalga have not been inactivated;
4) The N-acetylglycosyltransferase I of said microalga has not been inactivated, preferably the N-acetylglycosyltranferases II, III, IV, V and VI, mannosidase II and glycosyltransferases of said microalga have not been inactivated;

The terms “Chloroplast Endoplasmic Reticulum” or “CER” used herein refer to the outermost membrane of the plastid that is continuous with the Endoplasmic Reticulum membrane to which ribosomes are attached. This CER membrane is only found in plastid interconnected with the Endoplasmic Reticulum and harboring four membranes in heterokonts, cryptophytes and haptophytes. (see Bolte et al. (2009) as disclosed previously, Apt et al. (2002) In vivo characterization of diatom multipartite plastid targeting signals, Journal of Cell Science, n^{o} 115, pp: 4061-4069).

Therefore, microalgae used herein for the production of a protein harboring a non-immunogenic “high mannose” pattern of glycosylation in the plastid are aquatic photosynthetic microorganisms having a Chloroplast Endoplasmic Reticulum, said microalgae being selected in the group comprising heterokonts, cryptophytes and haptophytes.

In a further embodiment, said microalga of the present invention and having a CER is selected in a group of genus comprising Phaeodactylum, Nitzschia, Skeletonema, Chaetoceros, Odontella, Amphiprora, Thalassiosira, Nannochloropsis, Emiliania, Pavlova, Isochrysis, Apistonema, Rhodomonas.

In a further embodiment of the present invention, the microalga having a CER is the diatom Phaeodactylum tricornutum.

The term “nucleic acid sequence” used herein refers to DNA sequences (e.g., cDNA or genomic or synthetic DNA) and RNA sequences (e.g., mRNA or synthetic RNA), as well as analogs of DNA or RNA containing non-natural nucleotide analogs, non-native internucleoside bonds, or both. Preferably, said nucleic acid sequence is a DNA sequence. The nucleic acid can be in any topological conformation, such as linear or circular.

“Operatively linked” promoter and terminator refers to a linkage in which the promoter is contiguous with the gene of interest, said gene being also contiguous with the terminator in order to control both the expression and transcriptional termination of said gene.

Examples of promoter that drives expression of a polypeptide in a transformed microalga according to the invention include, but are not restricted to, endogenous nuclear promoters such as the promoter of the fucoxanthin-chlorophyll protein A (pFcpA) (GenBank accession number AF219942 from 1 pb to 142 pb, SEQ ID No38) and the promoter of the fucoxanthin-chlorophyll protein B gene (pFcpB) (GenBank accession number AF219942 from 848 pb to 1092 pb, SEQ ID No39) from Phaeodactylum tricornutum and exogenous promoters such as the promoter 35S (pCaMV35S) from the cauliflower mosaic virus (GenBank accession number AF502128 from 25 pb to 859 pb, SEQ ID No40), the promoter of the Nopaline Synthase (pNOS) from Agrobacterium tumefaciens (GenBank accession number X01077, SEQ ID No41).

Examples of terminator enabling the transcription termination of a gene in a transformed microalga according to the invention include, but are not restricted to, endogenous nuclear terminators such as the terminator of the fucoxanthin-chlorophyll protein A (tFcpA) (GenBank accession number AF219942 from 1468 pb to 1709 pb, SEQ ID No42) from Phaeodactylum tricornutum and exogenous terminators such as the terminator of the Nopaline Synthase (tNOS) from Agrobacterium tumefaciens (GenBank accession number AF502128 from 2778 pb to 3030 pb, SEQ ID No43).

Transformation of microalgae can be carried out by conventional methods such as microparticles bombardment, electroporation, glass beads, polyethylene glycol (PEG), silicon carbide whiskers, or use of viruses or agrobacterium (see Leon and Fernandez (2007) Transgenic microalgae as green cell factories, Landes Bioscience).

In a preferred embodiment, the transformation of a microalga according to the invention is a nuclear transformation for the integration of a foreign nucleic acid sequence into the nuclear genome of said microalga, wherein said nucleic acid sequence may be introduced via a plasmid, virus sequences, double or single strand DNA, circular or linear DNA.

It is generally desirable to include into each nucleotide sequences used for genetic transformation at least one selectable marker to allow selection of microalgae that have been transformed.

Examples of such markers for the transformation of microalga according to the invention are antibiotic resistant genes such as the bleomycin resistance gene (sh ble) enabling resistance to zeocin, nourseothricin resistance genes (nat or sat-1) enabling resistance to nourseothricin, hygromycin phosphotransferase II gene (hptII) enabling resistance to hygromycin or Aminoglycoside-O-phosphotransferase VIII gene (AphVIII) enabling resistance to paromomycin (see also León and Fernández (2007) as disclosed previously).

Alternatively, complementation of auxotrophic mutant strains of microalgae using genes that enable the reversion to prototrophy offers a selection system without the need of antibiotics. Examples of such complementation system include amino acid auxotrophs.

After transformation of microalgae, transformants producing the desired proteins accumulating into the plastidial stroma are selected by the above-mentioned selection methods. Alternatively, analysis of the protein to be produced can also be used as a mean of selection on whole microalgae by one or more conventional methods comprising: fluorimeter or immunocytochemistry by exposing cells to an antibody having a specific affinity for the desired protein. This type of selection can also be carried out on disrupted cells by one or more conventional methods comprising: enzyme-linked immunosorbent assay (ELISA), mass spectroscopy such as MALDI-TOF-MS, ESI-MS chromatography or spectrophotometer.

The term “signal peptide” as used herein refers to an amino acid sequence located at the amino terminal end of a polypeptide and which mediates the co-translational transport of said polypeptide across the CER membrane and into the ER lumen where cleavage of the signal peptide finally occurs. This signal peptide is typically 15-30 amino acids long, and presents a 3 domains structure (von Heijne (1990) The signal Peptide, Journal of Membrane Biology, n^{o} 115, pp: 195-201; Emanuelsson et al. (2007) Locating proteins in the cell using TargetP, SignalP and related tools. Nature Protocols, n^{o} 2, pp: 953-971), which are as follows:
(i) an N-terminal region (n-region) containing positively charged amino acids, such as Arginine (R), Histidine (H) or Lysine (K);
(ii) a central hydrophobic region (h-region) of at least 6 amino acids containing hydrophobic amino acids such as Alanine (A), Cysteine (C), Glycine (G), Isoleucine (I), Leucine (L), Methionine (M), Phenylalanine (F), Proline (P), Tryptophan (W) or Valine (V); and
(iii) a C-terminal region (c-region) of polar uncharged amino acids such as Asparagine (R), Glutamine (Q), Serine (S), Threonine (T) or Tyrosine (Y). Said C-region often contains a helix-breaking proline or glycine that helps define a cleavage site. Small uncharged residues in positions −3 and −1 (defined as the number of residue before the cleavage site) are usually requires for an efficient cleavage by signal peptidase following the translocation across the endoplasmic reticulum membrane (von Heijne (1990) as disclosed previously; Verner and Schatz (1988) Protein translocation across membranes, Science, n^{o} 241, pp: 1307-1313).

A person skilled in the art is able to simply identify a signal peptide in an amino acid sequence, for example by using the SignalP 3.0 program (accessible on line at http://www.cbs.dtu.dk/services/SignalP/) which predicts the presence and location of signal peptide cleavage sites in amino acid sequences from different organisms by using two different models: the Neural networks and the Hidden Markov models (Emanuelsson et al. (2007) as disclosed previously).

The term “transit peptide” as used herein refers to an amino acid sequence that is contiguous to the amino terminal end of a polypeptide and which is sufficient to mediate the transport of said polypeptide through the 3 innermost membranes of the plastid and into the stroma of the plastid where the transit peptide is then cleaved off.

Transit peptides show a broad heterogeneity in term of structural features. They vary in length between 8-150 amino acids.

Preferably, the transit peptide according to the invention comprises less than 60 amino acids.

Transit peptides targeting proteins into the plastidial stroma comprise an aromatic residue such as phenylalanine, tryptophan, or tyrosine at position +1 of the transit peptide relative to the signal peptide's predicted cleavage site.

Transit peptides also possess a high content of hydroxylated residues as well as an overall positive charge due to the presence of basic, positively charged amino acids such as lysine, arginine or histidine and a low content of acidic, negatively charged residues such as aspartate and glutamate (Jarvis (2008) Targeting of nucleus-encoded proteins to chloroplasts in plants, New Phytologist, n^{o} 179, pp: 257-285). The overall charge of said transit peptide can be calculated as the number of basic, positively charged residues minus the number of acidic, negatively charged residues.

In a preferred embodiment, said transit peptide comprises at least 10% of hydroxylated residues, more preferably at least 13% of hydroxylated residues.

In a preferred embodiment, the overall charge of said transit peptide is at least +1, more preferably at least +2.

A person skilled in the art is able to simply identify a transit peptide in an amino acid sequence, for example by using the TargetP program (accessible on line at http://www.cbs.dtu.dk/services/TargetP/) or iPSORT program (accessible on line at http://ipsort.hgc.jp/). Alternatively, the Nectar program can also be used for the prediction of the whole bipartite topogenic signal sequence in heterokonts (accessible on line at http://www.sb-roscoff.fr/hectar/).

The term “bipartite topogenic signal” sequence or “BTS” as used herein refers to an amino acid sequence that is contiguous to the amino terminal end of a polypeptide and composed of a signal peptide adjoining a transit peptide. Said BTS sequence leads to the co-translational import in the Endoplasmic Reticulum further followed by the transport of the protein in the plastid of the microalga, said protein according to the invention harboring a non-immunogenic “high mannose” pattern of glycosylation. The cleavage of the signal peptide of the aforementioned bipartite topogenic signal sequence in the Endoplasmic Reticulum leads to the exposure of the transit peptide for further targeting of the protein to be produced in the plastid of the transformed microalga.

The bipartite topogenic signal sequence can be identified by bioinformatic analyses performed on protein sequences of heterokonts, cryptophytes or haptophytes from publicly available databases such as the US Department of Energy Joint Genome Institute (JGI, http://www.jgi.doe.gov/). The putative protein sequences are screened for the presence of signal peptides using SignalP 3.0. Based on the program's prediction cleavage site, retained sequences are processed to remove amino acids corresponding to the signal peptides and further screened for the presence of transit peptide using TargetP or iPSORT. The transit peptides identified are checked for their overall net charge, the content of hydroxylated residues and the occurrence of an aromatic amino acids at position +1 as previously described. Bipartite topogenic signal sequences can then be retrieved by multi-steps analysis and used in-frame for the targeting of proteins to be produced.

In a preferred embodiment, the invention relates to the use of a transformed microalga for the production of at least one protein harboring a “high mannose” pattern of glycosylation in the plastid of said transformed microalga, preferably a non-immunogenic “high mannose” pattern of glycosylation, wherein
1) said transformed microalga has a Chloroplast Endoplasmic Reticulum (CER);
2) said microalga is transformed with a nucleic acid sequence operatively linked to a promoter, said nucleic acid sequence encoding an amino acid sequence comprising:
   (i) An amino-terminal bipartite topogenic signal (BTS) sequence composed of at least a signal peptide followed by a transit peptide; and
   (ii) The sequence of said protein.
3) the xylosyltransferases and fucosyltransferases of said microalga have not been inactivated;
4) The N-acetylglycosyltransferase I of said microalga has not been inactivated, preferably the N-acetylglycosyltranferases II, III, IV, V and VI, mannosidase II and glycosyltransferases of said microalga have not been inactivated;
5) Said BTS sequence leads to the co-translational import of the protein into the Endoplasmic Reticulum followed by the transport of said protein in the plastid of the microalga, said protein harboring a “high mannose” pattern of glycosylation.

In another preferred embodiment, the bipartite topogenic signal sequence is selected in the group comprising:
(a) the amino acid sequence set forth in SEQ ID No1 from the Light Harversting Complex Protein 11 LHCP11 of Guillardia theta;
(b) the amino acid sequence set forth in SEQ ID No2 from the chloroplast ATPase Gamma subunit (AtpC) protein of P. tricornutum;
(c) the amino acid sequence set forth in SEQ ID No3 from the triose Phosphate/Phosphate Translocator (Tpt1) protein of P. tricornutum;
(d) the amino acid sequence set forth in SEQ ID No4 from the Fucoxanthin-chlorophyll a-c binding protein D (FcpD) protein of P. tricornutum;
(e) the amino acid sequence set forth in SEQ ID No5 from the Fructose-1,6-bisphophatase (FBPC4) protein of P. tricornutum;
(f) the amino acid sequence set forth in SEQ ID No6 from the Oxygen-evolving Enhancer 1 (OEE1) protein of P. tricornutum;

wherein the nucleic acid sequence coding for the bipartite topogenic signal is in-frame with the nucleic acid sequence coding for the recombinant protein to be produced. In a most preferred embodiment, the bipartite topogenic signal sequence is selected in the group comprising:
(a) the amino acid sequence set forth in SEQ ID No2;
(b) the amino acid sequence set forth in SEQ ID No3;
(c) the amino acid sequence set forth in SEQ ID No1;

The term “polypeptide” or “protein” as used herein refers to an amino acid sequence comprising amino acids which are linked by peptide bonds. A polypeptide may be monomeric or polymeric. Furthermore, a polypeptide may comprise a number of different domains each of which has one or more distinct activities.

The term “glycosylated polypeptide/protein” or “glycoprotein” as used herein refers to a protein with N-glycosylation.

The protein to be produced according to the invention is a protein harboring a “high mannose” pattern of glycosylation.

The term “N-glycan” as used herein refers to a N-linked oligosaccharide, e.g., one that is attached by a linkage between the N-acetylglucosamine of said oligosaccharide and an asparagine residue at a site of N-glycosylation.

The term “site of N-glycosylation” refers to the asparagine residues of the consensus sequences Asn-X-Ser/Thr, when X is different than proline and aspartic acid, of a protein.

The expressions “high mannose pattern of N-glycosylation” in reference to a protein or “high mannose N-glycosylated protein” refer to a protein harboring high mannose N-linked oligosaccharides, i.e. a protein having on each occupied site of N-glycosylation a glycan composed of 5 to 9 mannose residues and at least one exposed mannose residue (terminal mannose residue).

Preferably, said protein comprises on each occupied N-glycosylation site a glycan composed of 6 to 9 mannose residues and at least one exposed mannose residue.

The term “occupied site of N-glycosylation” refers to a site of N-glycosylation harboring a N-glycan, i.e. to an asparagine residues of the consensus sequences Asn-X-Ser/Thr, when X is different than proline and aspartic acid, harboring oligosaccharides.

Preferably, the protein to be produced according to the invention has at least 5 to 9 mannoses residues, most preferably from 6 to 9 mannose residues on oligosaccharides located at the level of the asparagine residues of the consensus sequences Asn-X-Ser/Thr, when X is different than proline and aspartic acid, of said protein.

Preliminary information about N-glycan of the recombinant protein can be obtained by affino- and immunoblotting analysis using specific probes such as lectins (ConA from Canavalia ensiformis; GNL from Galanthus nivalis; HHL from Hippeastrum hybrid; ECA from Erythrina cristagalli; SNA from Sambucus nigra; MAA from Maackia amurensis . . . ) and specific N-glycan antibodies (anti-β(1,2)-xylose; anti-α(1,3)-fucose; anti-Neu5Gc, anti-Lewis . . . ). To investigate the detailed N-glycan profile of recombinant protein, N-linked oligosaccharides is released from the protein in a non specific manner using enzymatic digestion or chemical treatment. The resulting mixture of reducing oligosaccharides can be profiled by HPLC and/or mass spectrometry approaches (ESI-MS-MS and MALDI-TOF essentially). These strategies, coupled to exoglycosidase digestion, enable N-glycan identification and quantification (see Dolashka et al. (2010) Glycan structures and antiviral effect of the structural subunit RvH2 of Rapana hemocyanin, Carbohydrate Research, n^{o} 345, pp:2361-2367).

In a preferred embodiment, the protein to be produced according to the invention is a protein harboring a non-immunogenic “high mannose” pattern of glycosylation.

The expression “non-immunogenic pattern of glycosylation” in reference to a protein to be produced according to the invention refers to a pattern of glycosylation which does not elicit an immune response in the human body. As an example, immunogenic pattern of glycosylation comprising β(1,2)-xylose, α(1,3)-fucose, N-glycolylneuraminic acid and/or galactose-α(1,3)-galactose on N-glycans may elicit immune response. Said non-immunogenic pattern of glycosylation of the protein according to the invention arises from the absence of transit of said protein through the Golgi apparatus in which immunogenic patterns are usually added by glycosyltransferases.

Preferably, a non-immunogenic pattern of glycosylation refers to a pattern of glycosylation not harboring any α(1,3)-fucose or β(1,2)-xylose on N-glycans, i.e. on oligosaccharides located at the level of the asparagine residues of the consensus sequences Asn-X-Ser/Thr, when X is different than proline and aspartic acid, of said protein.

In a preferred embodiment, the protein produced in the plastid of microalga according to the invention presents a homogenous pattern of glycosylation.

The term “homogenous” refers to a pattern of glycosylation comprising a majority of “high mannose” N-glycans.

Advantageously, a homogenous pattern of glycosylation comprises at least 70%, preferably at least 80% or at least 90%, and most preferably at least 95% of “high mannose” N-glycans.

In another most preferred embodiment, the protein presenting a homogenous pattern of glycosylation according to the invention does not comprise galactose, sialic acid, fucose and/or xylose on N-glycans.

The determination of the homogeneity of a pattern of glycosylation can be realized by analyzing N-glycans as described previously.

Preferably, the spectrum of released N-glycans from the protein produced according to the invention does not comprise peaks corresponding to oligosaccharides having at least one of the following sugar residues: galactose, sialic acid, fucose, xylose.

In a preferred embodiment, the protein according to the invention is a heterologous protein.

The term “heterologous”, with reference to a protein, means an amino acid sequence which does not exist in the corresponding microalga before its transformation. It is intended that the term encompasses proteins that are encoded by wild-type genes, mutated genes, and/or synthetic genes.

In a still preferred embodiment, said heterologous protein which is produced and transported in the plastid of a transformed microalga according to the invention can be a protein used for therapeutic purposes, wherein said protein can be of viral or animal origin. Preferably, said animal polypeptide is of mammalian origin. Most preferably, said mammalian polypeptide is of human origin.

In a preferred embodiment, the protein according to the invention is a protein selected in the group comprising human lysosomal enzymes, viral envelope glycoproteins or viral envelope glycoprotein's fragments, antibodies or antibody's fragments and derivatives thereof.

The term “lysosomal enzyme” refers to hydrolases that are naturally produced by the human body having an enzymatic activity in the lysosome organelle. Said enzyme is responsible for breaking down complex chemicals, macromolecules or other materials contained in the lysosome. Deficiencies of such enzymes are responsible for the accumulation of lysosomal metabolites leading to pathologies known as lysosomal storage disorders. Examples of such deficient enzymes and related diseases include:
α-fucosidase (Fucosidosis)
α-galactosidase A (Fabry disease)
α-L-iduronidase (Hurler syndrome; Mucopolysaccharidosis type I)
Iduronate-2-sulphatase (Hunter syndrome; Mucopolysaccharidosis type II)
Arylsulfatase B (Maroteaux-Lamy syndrome; Mucopolysaccharidosis type VI)
Acid α-mannosidase (α-mannosidosis)
α-neuraminidase (sialidosis)
Acid α-glucosidase (Pompe disease)
Acid β-galactosidase (GM1 gangliosidosis)
Acid β-glucosidase (Gaucher disease)
β-glucuronidase (Sly syndrome; MPS VII)
Acid β-mannosidase (β-mannosidosis)
Acid Sphingomyelinase (Niemann-Pick disease)
Lysosomal acid lipase (Wolman disease)

Examples of lysosomal enzymes to be produced using the present invention include α-galactosidase A, α-fucosidase, α-L-iduronidase, iduronate-2-sulfatase, arylsulfatase B, acid sphingomyelinase, acid α-mannosidase, acid α-glucosidase, α-neuraminidase, acid β-galactosidase, acid β-glucosidase, β-glucuronidase, acid β-mannosidase and lysosomal acid lipase.

Lysosomal enzymes produced according to the invention harbor high-mannose oligosaccharides and therefore can be taken up by cells through their mannose receptors to replace deficient enzymes in the so-called enzyme replacement therapy (ERT).

The term “Viral envelope glycoprotein” refers to a glycosylated protein included in the viral envelope covering the protein capsid of the virion particle. Said viral envelope glycoprotein is located on the surface of the envelope enabling the binding of the virion particle onto receptors of host cell leading ultimately to entry of the virus into the cell.

Examples of such viral envelope glycoproteins are the precursor gp160 and its processed forms gp120 and gp41 proteins from type 1 human immunodeficiency virus (HIV), E1 and E2 proteins from hepatitis C virus, the E protein from the dengue virus and west nile virus, the GP protein from Ebola virus.

The term “viral envelope glycoprotein's fragments” as used herein refers to fragments of said envelope glycoprotein.

An “antibody” is an immunoglobulin molecule corresponding to a tetramer comprising four polypeptide chains, two identical heavy (H) chains (about 50-70 kDa when full length) and two identical light (L) chains (about 25 kDa when full length) inter-connected by disulfide bonds. Light chains are classified as kappa and lambda. Heavy chains are classified as gamma, mu, alpha, delta, or epsilon, and define the antibody's isotype as IgG, IgM, IgA, IgD, and IgE, respectively. Each heavy chain is comprised of an amino-terminal heavy chain variable region (abbreviated herein as HCVR) and a heavy chain constant region. The heavy chain constant region is comprised of three domains (CH1, CH2, and CH3) for IgG, IgD, and IgA; and 4 domains (CH1, CH2, CH3, and CH4) for IgM and IgE. Each light chain is comprised of an amino-terminal light chain variable region (abbreviated herein as LCVR) and a light chain constant region. The light chain constant region is comprised of one domain, CL. The HCVR and LCVR regions can be further subdivided into regions of hypervariability, termed complementarity determining regions (CDRs), interspersed with regions that are more conserved, termed framework regions (FR). Each HCVR and LCVR is composed of three CDRs and four FRs, arranged from amino-terminus to carboxy-terminus in the following order: FR1, CDR1, FR2, CDR2, FR3, CDR3, FR4. The assignment of amino acids to each domain is in accordance with well-known conventions. The functional ability of the antibody to bind a particular antigen depends on the variable regions of each light/heavy chain pair, and is largely determined by the CDRs.

The term “antibody”, as used herein, refers to a monoclonal antibody per se. A monoclonal antibody can be a human antibody, chimeric antibody and/or humanized antibody.

Antibodies to be produced according to the invention are for example recombinant IgG antibodies having enhanced antibody dependent cell-mediated cytotoxicity (ADCC).

The term “antibody fragments” as used herein refers to antibody fragments that bind to the particular antigens of said antibody. For example, antibody fragments capable of binding to particular antigens include Fab (e.g., by papain digestion), Fab′ (e.g., by pepsin digestion and partial reduction) and F(ab′)2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention. Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)2 heavy chain portion can be designed to include DNA sequences encoding the CH₁ domain and/or hinge region of the heavy chain. The various portions of antibodies can be joined together chemically by conventional techniques, or can be prepared as a contiguous protein using genetic engineering techniques.

As used herein, the term “derivative” refers to a polypeptide having a percentage of identity of at least 90% with the complete amino acid sequence of any of the protein disclosed previously and having the same activity.

Preferably, a derivative has a percentage of identity of at least 95% with said amino acid sequence, and preferably of at least 99% with said amino acid sequence.

As used herein, “percentage of identity” between two amino acids sequences, means the percentage of identical amino acids, between the two sequences to be compared, obtained with the best alignment of said sequences, this percentage being purely statistical and the differences between these two sequences being randomly spread over the amino acids sequences. As used herein, “best alignment” or “optimal alignment”, means the alignment for which the determined percentage of identity (see below) is the highest. Sequences comparison between two amino acids sequences are usually realized by comparing these sequences that have been previously aligned according to the best alignment; this comparison is realized on segments of comparison in order to identify and compare the local regions of similarity. The best sequences alignment to perform comparison can be realized by using computer softwares using algorithms such as GAP, BESTFIT, BLAST P, BLAST N, FASTA, TFASTA in the Wisconsin Genetics software Package, Genetics Computer Group, 575 Science Dr., Madison, Wis. USA. To get the best local alignment, one can preferably used BLAST software, with the BLOSUM 62 matrix, preferably the PAM 30 matrix. The identity percentage between two sequences of amino acids is determined by comparing these two sequences optimally aligned, the amino acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences.

In a most preferred embodiment of the invention, proteins to be produced according to the invention are selected in the group comprising the sequences disclosed in Table I or derivatives thereof, wherein said protein sequences are fused downstream of a bipartite topogenic signal peptide.

TABLE I

| | CDS SEQ | Accession | |
|---|---|---|---|
| PROTEIN | ID N° | number (Protein) | Comments |
| β-glucocerebrosidase = | SEQ ID N° 7 | AAA35873 | Lysosomal enzyme |
| Acid β-glucosidase | | | |
| α-Galactosidase A | SEQ ID N° 8 | NP_000160 | Lysosomal enzyme |
| Alglucosidase = | SEQ ID N° 9 | NP_000143 | Lysosomal enzyme |
| Acid α-glucosidase | | | |
| α-L-iduronidase | SEQ ID N° 10 | NP_000194 | Lysosomal enzyme |
| Iduronate 2-sulfatase | SEQ ID N° 11 | NP_000193 | Lysosomal enzyme |
| Arylsulfatase B | SEQ ID N° 12 | NP_000037 | Lysosomal enzyme |
| Acid Sphingomyelinase | SEQ ID N° 13 | NP_000534 | Lysosomal enzyme |
| Lysosomal acid lipase | SEQ ID N° 14 | NP_001121077 | Lysosomal enzyme |
| GP120 | SEQ ID N° 15 | NP_579894 | Envelope |
| | | | glycoprotein |
| | | | from Human |
| | | | Immunodeficiency |
| | | | Virus 1 |
| GP41 | SEQ ID N° 16 | NP_579895 | Envelope |
| | | | transmembrane |
| | | | glycoprotein |
| | | | from Human |
| | | | Immunodeficiency |
| | | | Virus 1 |
| E1 protein | SEQ ID N° 17 | From aa 192 to 383 | Envelope |
| | | of the polyprotein | glycoprotein from |
| | | P27958 | Hepatitis C Virus |
| E2 protein | SEQ ID N° 18 | From aa 384 to 746 | Envelope |
| | | of the polyprotein | glycoprotein from |
| | | P27958 | Hepatitis C Virus |
| E protein | SEQ ID N° 19 | From aa 281 to 775 | Envelope |
| | | of the polyprotein | glycoprotein from |
| | | ADO97105 | Dengue virus 1 |
| E protein | SEQ ID N° 20 | From aa 291 to 791 | Envelope |
| | | of the polyprotein | glycoprotein from |
| | | ADL27981 | West Nile Virus |
| Spike glycoprotein | SEQ ID N° 21 | ACI28632 | Envelope |
| precursor | | | glycoprotein from |
| | | | Ebola virus |
| immunoglobulin | SEQ ID N° 22 | CAC20454 | Gamma 1 |
| heavy chain | SEQ ID N° 23 | CAC20457 | Gamma 4 |
| constant region gamma | | | |
| Immunoglobulin | SEQ ID N° 24 | AAA59127 | |
| Variable Heavy Chain | | | |
| Immunoglobulin Kappa | SEQ ID N° 25 | CAA09181 | |
| light Chain (VL + CL) | | | |

Still most preferably, said glycosylated protein is the β-glucocerebrosidase as encoded by the amino acid sequence set forth in SEQ ID No7, the α-Galactosidase A as encoded by the amino acid sequence set forth in SEQ ID No8, the α-L-iduronidase as encoded by the amino acid sequence set forth in SEQ ID No10, the Alglucosidase as encoded by the amino acid sequence set forth in SEQ ID No9, the Acid Sphingomyelinase as encoded by the amino acid sequence set forth in SEQ ID No13, the GP120 (HIV) as encoded by the amino acid sequence set forth in SEQ ID No15, the E1 (HCV) as encoded by the amino acid sequence set forth in SEQ ID No17 and the E2 (HCV) as encoded by the amino acid sequence set forth in SEQ ID No18.

According to the invention, xylosyltransferases and fucosyltransferases from the microalga used for the production of a protein harboring a non-immunogenic “high mannose” pattern of N-glycosylation have not been inactivated.

The expression “not to have been inactivated” with reference to an enzyme means that the activity of said enzyme in the microalga of the invention has neither been modified nor suppressed by its transformation.

Xylosyltransferases are enzymes having an activity of adding p(1,2)-linked xyloses on N-glycans of glycoproteins in the Golgi apparatus.

Fucosyltransferases are enzymes having an activity of adding α(1,3)-linked fucoses on N-glycans of glycoproteins in the Golgi apparatus.

Still according to the invention, the N-acetylglucosaminyltransferase I has not been inactivated.

The N-acetylglucosaminyltransferase I is capable of adding an N-acetylglucosamine (GlcNac) residue to Man₅GlcNac₂ to produce GlcNacMansGlcNac₂ in the Golgi apparatus.

Preferably, the N-acetylglucosaminyltranferases II, III, IV, V and VI, the mannosidase II and the glycosyltransferases of the glycosylation pathway of said microalga have not been inactivated.

Glycosyltransferases comprise galactosyltransferases, fucosyltransferases, xylosyltransferases and sialyltransferases.

Another object of the invention is a vector comprising a nucleic acid sequence operatively linked to a promoter, wherein said nucleic acid sequence encodes an amino acid sequence comprising:
i) a bipartite topogenic signal (BTS) composed of at least a signal peptide and a transit peptide as defined previously, and
ii) the sequence of a protein to be produced as defined previously.

The term “vector” refers to any vehicle capable of facilitating the transfer of a nucleic acid sequence in a microalga. Said term “vector” encompasses without limitation the plasmids, cosmids, phagemids or any other vehicle derived from viral or proteic sources which have been manipulated for the insertion or incorporation of a nucleic acid sequence into a microalga.

In a preferred embodiment, the vector according to the invention also comprises a nucleic acid sequence encoding a selectable marker operatively linked to a promoter as defined previously. Alternatively, a nucleic acid sequence encoding a protein enabling the restoration of prototrophy operatively linked to a promoter can be included in the vector of the present invention.

Another object of the invention is a microalga comprising a nucleic acid sequence operatively linked to a promoter, wherein said nucleic acid sequence encodes an amino acid sequence comprising:
i) a bipartite topogenic signal (BTS) composed of at least a signal peptide and a transit peptide as defined previously, and
ii) the sequence of a protein as defined previously.

Another embodiment of the invention discloses a microalga comprising a vector as defined previously.

Another object of the invention is a method for producing at least one protein in a transformed microalga having a Chloroplast Endoplasmic Reticulum (CER) as defined previously, said method comprising the steps of:

1) Culturing said transformed microalga;

2) Harvesting the plastid of said transformed microalga;

3) Purifying said protein from said plastid.

The culture of the transformed microalga according to the invention can be carried out by conventional methods of culture according to the specie of the microalga which has been selected for the transformation and production of proteins. A protocol that can be used for the cultivation of microalgae of the present invention is given in the example section.

Method for isolation of plastid from transformed microalga according to the invention includes, but is not limited to, the use of density gradient centrifugation. Said method includes an initial step to release the microalgal cell content by homogenization in a medium containing sorbitol followed by a purification step on a 40% Percoll continuous gradient. Alternatively, the method of “cell disruption” leading to the release of the whole intracellular content can be used for the releasing of the plastid content. A method for cell disruption of microalgae of the present invention by sonication is given in the example section.

The purification of the protein to be produced according to the invention can be carried out by chromatography. Such method includes the use of filtration followed by concanavalin A chromatography to specifically purified glycoproteins. Gel filtration and ion-exchange chromatography can also be used to purify further the recombinant polypeptide.

In another embodiment, the protein of the invention can be fused to an amino- or carboxy-terminal Tag for the purpose of purification of such protein. The term “Tag” as used herein refers to an amino acid sequence fused to a protein. An example of Tag include the histidine tag composed of six histidine residues that can be purified as described in the example section.

In another embodiment, the method for producing a glycoprotein stored in the plastid of said transformed microalga comprises a former step of transforming said microalga with a nucleic acid sequence operatively linked to a promoter as defined previously.

In another embodiment, the method for producing a glycoprotein stored in the plastid of said transformed microalga comprises a former step of transforming said microalga with a vector as defined previously.

In another embodiment of the invention, the method for producing a protein stored in the plastid of a transformed microalga further comprises a step 4) of determining the N-glycosylation pattern of said protein and selecting the protein harboring a “high mannose” pattern of N-glycosylation.

Preliminary information about N-glycosylation of the recombinant protein accumulated in the plastid can be obtained by affinodetection analysis using specific probes such as lectins (ConA from Canavalia ensiformis; GNL from Galanthus nivalis; HHL from Hippeastrum hybrid; ECA from Erythrina cristagalli; SNA from Sambucus nigra; MAA from Maackia amurensis . . . ). Lack of β(1,2)-xylose, α(1,3)-fucose, Neu5Gc and Lewis epitopes can be assessed by immunoblotting analysis using specific N-glycans antibodies (anti-β(1,2)-xylose; anti-α(1,3)-fucose; anti-Neu5Gc; anti-Lewis . . . ). To investigate the detailed N-glycan profile of recombinant polypeptide, N-linked oligosaccharides is released from the polypeptide in a non specific manner using enzymatic digestion or chemical treatment. The resulting mixture of reducing oligosaccharides can be profiled by HPLC and/or mass spectrometry approaches (ESI-MS-MS and MALDI-TOF essentially). These strategies, coupled to exoglycosidase digestion, enable N-glycan identification and quantification (Séveno et al. (2008) Plant N-glycan profiling of minute amounts of material, Analytical Biochemistry, n^{o} 379, pp: 66-72; Dolashka et al. (2010) as disclosed previously).

In a preferred embodiment, the method of producing proteins harboring a “high mannose” N-glycosylation pattern in the plastid of transformed microalgae leads to the transport of at least 70%; preferably 80% and most preferably 90% of said proteins in the plastid.

Said quantity of transported proteins in the stroma of the plastid of said microalgae can be determined using the ratio between the overall quantity of the recombinant protein and the quantity of said protein within the plastid. The overall quantity of recombinant protein is being defined as the sum of intracellular and extracellular quantity of said recombinant protein. Intracellular content of the aforementioned protein can be obtained by cell disruption method while plastidial content of said protein is obtained by purification of said organelle as described previously. Quantities of the recombinant protein can be determined on the intracellular, extracellular and plastidial fractions by enzyme-linked immunosorbent assay (ELISA) on fractions. The percentage of protein stored in the plastid can be calculated as follow:

% plastidial=(Q_{plastidial}×100)/(Qᵢₙₜₑᵣₙₐₗ+Qₑₓₜₑᵣₙₐₗ)

### Wherein:

Q_{plastidial} is the quantity of the recombinant protein to be produced detected in the fraction of purified plastid.
Qᵢₙₜₑᵣₙₐₗ is the quantity of the recombinant protein to be produced detected in the intracellular fraction.
Qₑₓₜₑᵣₙₐₗ is the quantity of the recombinant protein to be produced detected in the extracellular fraction.
% plastidial is the percentage of the recombinant protein to be produced accumulated within plastid.

Another object of the invention is a protein harboring a non-immunogenic <<high mannose>> pattern of glycosylation and produced according to the method as defined previously.

Another object of the invention is a pharmaceutical composition comprising a protein harboring a <<high mannose>> pattern of glycosylation and produced according to the method of the invention.

Advantageously, said composition is used as a vaccine for inducing potent antigenic response.

In the following examples, the invention is described in more detail with reference to methods. Yet, no limitation of the invention is intended by the details of the examples. Rather, the invention pertains to any embodiment which comprises details which are not explicitly mentioned in the examples herein, but which the skilled person finds without undue effort.

### EXAMPLES

### Example 1

### Targeting of a Nuclear-Encoded Glycosylated Chimeric Protein into the Plastid of Phaeodactylum tricornutum

To test the ability of a nuclear-encoded protein to be glycosylated, targeted and stored into the plastid, Phaeodactylum tricornutum (P. tricornutum) was transformed with a plasmid containing a 54 amino acids bipartite topogenic signal sequence of the phosphoenolpyruvate/phosphate translocator (Tpt1) from P. tricornutum fused in-frame with a sequence coding for a chimeric protein composed of the mature murine erythropoietin (EPO) and the enhanced green fluorescent protein (eGFP) (SEQ ID No26). The chimeric protein is composed of 166 amino acids corresponding to the mature EPO protein lacking its native 26 amino acids signal peptide followed by the PreScission protease cleavage site (LEVLFQGP) and 239 amino acids corresponding to the green fluorescent protein. The chimeric protein obtained contains 3 potential N-glycosylation sites within the EPO sequence.

a) Standard Culture Conditions of Phaeodactylum tricornutum

The diatom Phaeodactylum tricornutum was grown at 20° C. under continuous illumination (280-350 μmol photons.m⁻².s⁻¹), in natural coastal seawater sterilized by 0.22 μm filtration. This seawater is enriched with nutritive Conway media with addition of silica (40 mg.L⁻¹ of sodium metasilicate). For large volume (from 2 litters to 300 liters), cultures were aerated with a 2% CO₂/air mixture to maintain the pH in a range of 7.5-8.1.

For genetic transformation, diatoms were spread on gelose containing 1% of agar. After concentration by centrifugation, the diatoms were spread on petri dishes sealed and incubated at 20° C. under constant illumination. Concentration of culture was estimated on Mallassez counting cells after fixation of microalgae with a Lugol's solution.

b) Expression Constructs for Genetic Transformation

The cloning vector pPha-T1 (GenBank accession number AF219942) includes sequences of P. tricornutum promoter fcpA (fucoxanthin-chlorophyll a/c-binding proteins A) upstream of a multiple cloning site followed by the terminator fcpA. It also contains a selection cassette with the promoter fcpB (fucoxanthin-chlorophyll a/c-binding proteins B) upstream of the coding sequence sh ble followed by the terminator fcpA (Zaslayskaia and Lippmeier (2000) transformation of the diatom Phaeodactylum tricornutum (Bacillariophyceae) with a variety of selectable marker and reporter genes, Journal of Phycology, n^{o} 36, pp 379-386). The sequence containing the bipartite topogenic signal sequence fused in-frame with the chimeric EPO-eGFP protein (nucleic acid sequence SEQ ID No26) was synthesized with the addition of EcoRI and HindIII restriction sites flanking the 5′ and 3′ ends respectively. Alternatively, a similar sequence containing a histidine tag at the carboxy-terminal (EPO-eGFP-HisTag) (nucleic acid sequence SEQ ID No27) was also synthesized with the addition of EcoRI and HindIII restriction sites flanking the 5′ and 3′ ends respectively.

The chimeric EPO-eGFP protein containing the pre-sequence of the ER luminal chaperone BiP and the diatom ER retention sequence DDEL (nucleic acid sequence SEQ ID No44 was synthesized with the addition of EcoRI and HindIII restriction sites flanking the 5′ and 3′ ends respectively. This construct was used as a control corresponding to a protein retained in the ER compartment (see Apt et al. (2002) as disclosed previously, Apt et al. (1995) The ER chaperone BiP from the diatom Phaeodactylum, Plant Physiology, n^{o} 109, p: 339).

After digestion by EcoRI and HindIII, each insert was introduced into the pPHA-T1 vector. As a control, an empty pPha-T1 vector lacking the EPO-eGFP coding sequence was used.

c) Genetic Transformation

The transformation was carried out by particles bombardment using the BIORAD PDS-1000/He apparatus modified (Thomas et al. (2001) A helium burst biolistic device adapted to penetrate fragile insect tissues, Jounal of Insect Science, n^{o} 1, pp 1-9).

Cultures of diatoms (P. tricornutum) in exponential growth phase were concentrated by centrifugation (10 minutes, 2150 g, 20° C.), diluted in sterile seawater, and spread on agar plate at 10⁸ cells per plate. The microcarriers are gold particles (diameter 0.6 μm). Microcarriers were prepared according to the protocol of the supplier (BIORAD). Parameters used for shooting were the following:
use of the long nozzle,
use of the stopping ring with the largest hole,
15 cm between the stopping ring and the target (diatom cells),
precipitation of the DNA by a solution containing 1.25 M CaCl₂ and 20 mM spermidine,
a ratio of 1.25 μg DNA for 0.75 mg gold particles per shot,
900 psi rupture disk with a distance of escape of 0.2 cm,
a vacuum of 30 Hg

Diatoms were incubated 24 hours before the addition of the antibiotic zeocin (100 μg.ml⁻¹) and were then maintained at 20° C. under constant illumination. After 1-2 weeks of incubation, individual clones were picked from the plates and inoculated into liquid medium containing zeocin (100 μg.ml⁻¹).

d) Microalgae DNA Extraction

Cells (5·10⁸) transformed by the various vectors were pelleted by centrifugation (2150 g, 15 minutes, 4° C.). Microalgae cells were incubated overnight at 4° C. with 4 mL of TE NaCl 1× buffer (Tris-HCL 0.1 M, EDTA 0.05 M, NaCl 0.1 M, pH 8). 1% SDS, 1% Sarkosyl and 0.4 mg.mL⁻¹ of proteinase K were then added to the sample, followed by an incubation at 40° C. for 90 minutes. A first phenol-chloroform-isoamyl alcohol extraction was carried out to extract an aqueous phase comprising the nucleic acids. RNA contained in the sample was eliminated by an hour incubation at 60° C. in the presence of RNase (1 μg.mL⁻¹). A second phenol-chloroform extraction was carried out, followed by a precipitation with ethanol. The pellet obtained was air-dried and solubilised into 200 μL, of ultrapure sterile water. Quantification of DNA was carried out by spectrophotometry (260 nm) and analysed by agarose gel electrophoresis.

e) Polymerase Chain Reaction (PCR) Analysis

The incorporation of the heterologous chimeric EPO-eGFP sequence in the genome of Phaeodactylum tricornutum was assessed by PCR analysis. The sequences of primers used for the PCR amplification were 5′-GTCTATATGAAGCTGAAGGG-3′ (SEQ ID No28) and 5′-GTGAGCAAGGGCGAGGAGC-3′ (SEQ ID No29) located in the EPO and eGFP sequence respectively. The PCR reaction was carried out in a final volume of 50 μl consisting of 1× PCR buffer, 0.2 mM of each dNTP, 5 μM of each primer, 20 ng of template DNA and 1.25 U of Taq DNA polymerase (Taq DNA polymerase, ROCHE). Thirty cycles were performed for the amplification of template DNA. Initial denaturation was performed at 94° C. for 4 min. Each subsequent cycle consisted of a 94° C. (1 min) melting step, a 55° C. (1 min) annealing step, and a 72° C. (1 min) extension step. Samples obtained after the PCR reaction were run on agarose gel (1%) stained with ethidium bromide.

Results revealed a single band at 276 bp for cells transformed with the constructs carrying the bipartite topogenic signal sequence fused to the chimeric EPO-eGFP (data not shown). No band was detected in cells transformed with the control vector. This result validated the incorporation of the exogenous gene in the genome of Phaeodactylum tricornutum.

f) Subcellular Localization of the Chimeric Protein

To investigate the sub-cellular localization of the chimeric protein EPO-eGFP, confocal microscopy was performed on wild-type and transformed cells of P. tricornutum. eGFP and chlorophyll fluorescence were excited at 488 nm, filtered and detected by two different photomultiplier tubes with bandwidths of 500-520 and 625-720 nm for eGFP (green channel) and chlorophyll fluorescence (red channel), respectively.

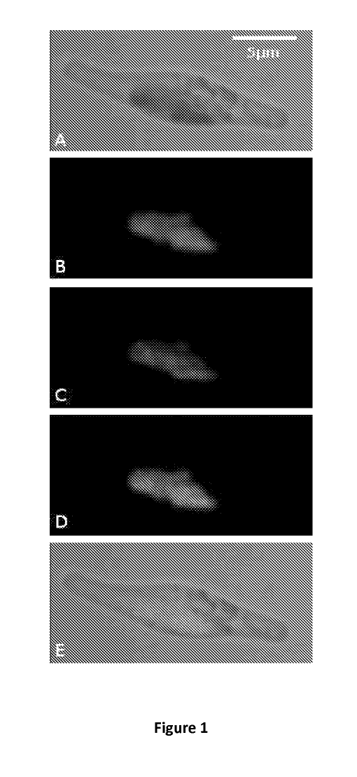

Confocal microscopy revealed the co-localization of the eGFP signal (FIG. 1.C.) with the position of the plastid as observed by bright field and autofluorescence of chlorophyll (FIGS. 1.A. and B.) as well as merged images (FIGS. 1.D. and E.). This result revealed that the use of the amino-terminal bipartite topogenic signal sequence from Tpt1 allowed the targeting of the chimeric protein EPO-eGFP to the chloroplast of P. tricornutum.

g) Immunoblotting Analysis

Aliquotes of wild-type and transformed cells of P. tricornutum culture at exponential phase of growth were collected and cells were separated from the culture medium by centrifugation (10 minutes, 2150 g, 20° C.). Cell pellets were resuspended in Tris-HCl 0.15 M pH 8, saccharose 15%, SDS 0.5%, PMSF 1 mM, protease inhibitor cocktail 1% (SIGMA) and sonicated for 30 min. Cell suspensions were centrifuged (60 minutes, 15000 g, 4° C.) to remove cell debris and supernatants were collected corresponding to the intracellular fraction.

Ten μL of intracellular fractions from plastid-targetd EPO-eGFP and ER-retained eGFP-EPO transformed cells as well as wild-type cells were separated by SDS-PAGE using a 12% polyacrylamide gel. The separated proteins were transferred onto nitrocellulose membrane and stained with Ponceau Red in order to control transfer efficiency. The nitrocellulose membrane was blocked overnight in milk 5% dissolved in TBS for immunodetection. Immunodetection was then performed using anti-EPO(R&D SYSTEMS, AF959) (1:500 in TBS-T containing milk 1% for 2 h at room temperature) or horseradish peroxidase-conjugated anti-eGFP (Santa Cruz, sc-9996-HRP) (1:2000 in TBS-T containing milk 1% for 2 h at room temperature). Membrane incubated with the anti-EPO antibody was then washed with TBS-T (6 times, 5 minutes, room temperature) and binding of the primary antibody was revealed upon incubation with a secondary horseradish peroxidase-conjugated rabbit anti-goat IgG (SIGMA-ALDRICH, A8919) (1:10000 in TBS-T containing milk 1% for 1.5 h at room temperature). All membranes were then washed with TBS-T (6 times, 5 minutes, room temperature) followed by a final wash with TBS (5 minutes, room temperature). Final development of the blots was performed by chemiluminescence method.

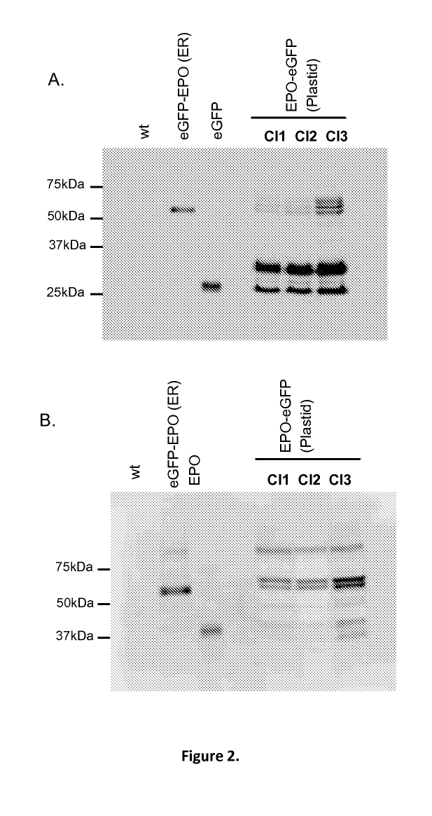

Samples from 3 transformed cell lines expressing EPO-eGFP fused to the bipartite topogenic signal sequence, 1 cell line expressing eGFP-EPO fused to the ER retention sequence, a wild-type cell line and murine EPO (R&D systems, 959ME) or eGFP (produced in E. colt) were run on a polyacrilamide gel in order to detect chimeric proteins by western blot using anti-GFP antibody and anti-EPO antibody. As depicted in FIGS. 2A and B, no band was visible in the sample from the wild-type (wt) cell line. Detection with anti-EPO or anti-eGFP antibodies showed a major band around 60 kDa in the sample corresponding to the ER-retained eGFP-EPO. Molecular weight of the corresponding amino acids sequence is around 49 kDa after the signal peptide is being cleaved. As murine EPO contains 3 N-glycosylation sites, the 60 kDa band suggested the glycosylation of the ER-retained eGFP-EPO.

For samples corresponding to plastid-targeted EPO-eGFP, comparative analysis of anti-EPO and anti-eGFP immunoblots revealed similar double bands around 60 kDa and 65 kDa. These double bands corresponding to plastid-targeted EPO-eGFP had a higher molecular weight when compared to the ER-retained EPO-eGFP suggesting heavier glycans. Other immunoreactive bands at various sizes were also detected which could account for unspecific detections or proteolysis (bands were detected at size similar to EPO or eGFP alone).

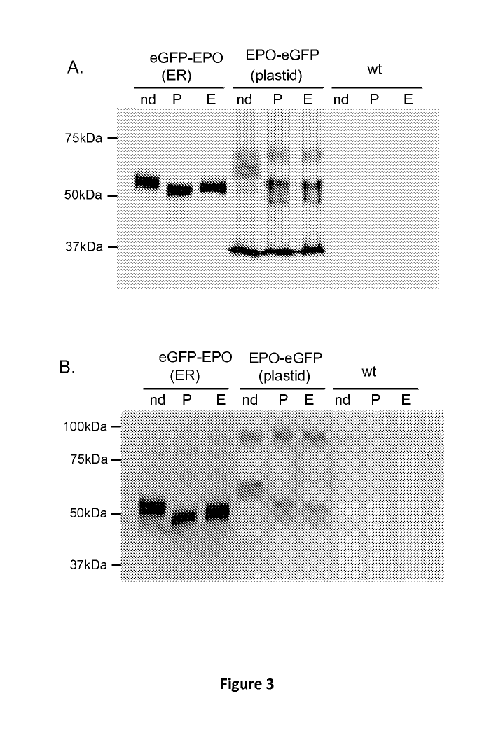

To further characterize glycans attached to plastid-targeted EPO-eGFP, deglycosylation assays were performed on the protein extracts prior to immunoblotting experiment using either peptide-N-glycosidase F (PNGase F, New England Biolabs) and endoglycosidase H (Endo H, New England Biolabs) according to manufacturer's recommendations. As depicted in FIGS. 3A and B, ER-retained eGFP-EPO was deglycosylated by PNGase F and endoglycosidase H. This result revealed that oligomannose, likely Man₆₋₉GlcNAc₂, are attached to EPO N-glycosylation sites as expected for ER resident proteins. Similar treatments performed on plastid-targeted EPO-eGFP also demonstrated the attachment of N-linked oligomannose. Altogether, these results indicated that plastid-targeted glycoproteins also contained oligomannose glycans (Man₆₋₉GlcNAc₂). Furthermore, the higher molecular weight observed when compared to ER resident proteins suggested a higher number of mannose residues on average for plastid-targeted glycoprotein.

h) Purification of the Chimeric Protein

The chimeric protein EPO-eGFP carrying the histidine tag is purified by chromatography method. Intracellular fractions from EPO-eGFP-HisTag as well as wild-type cells (control) are prepared as previously described. Both fractions are filtered using a membrane filter of 0.22 μm pore size, concentrated 10 times, and buffer-exchanged with 20 mM Tris, pH 9 containing 5 mM imidazole using a concentration device (MILLIPORE, Amicon Ultra-15, 3 kDa). Purification is performed using the AKTA FPLC system (GE Healthcare) and a Ni Sepharose column (GE Healthcare). The column is equilibrated with 20 mM Tris, pH 9.0 buffer containing 5 mM imidazole and the sample is then loaded. The column is washed with buffer containing 10 mM imidazole followed by elution with buffer containing 200 mM imidazole. The peak is collected and loaded on a Sephadex G-50 column equilibrated with 5 mM sodium phosphate buffer, pH 7.4. The desalted protein is collected, concentrated using a concentration device (MILLIPORE, Amicon Ultra-15, 3 kDa) and analysed by immunoblotting.

i) Structural Characterization of N-Linked Glycans of the Chimeric Protein

The chimeric EPO-eGFP carrying the histidine tag purified by chromatography method is subjected to enzymatic deglycosylation using PNGase F or Endo H in order to release N-linked glycans. Released glycans are analyzed by mass spectrometry as described by Dolashka et al., (2010) Glycan structures and antiviral effect of the structural subunit RvH2 of Rapana hemocyanin, Carbohydr Res, 345:2361-2367.

### Example 2

### Targeting of Nuclear-Encoded Human Lysosomal Enzyme into the Plastid of Phaeodactylum Tricornutum

The β-glucocerebrosidase (GBA) is an enzyme naturally targeted to the lysosomal compartments of human cells. Enzymatic deficiency leads to the accumulation of glucocerebroside in macrophages causing Gaucher's disease. Treatments include enzyme replacement therapy based on the delivery of intravenously injected recombinant β-glucocerebrosidase. The FDA-approved drug is produced in Chinese Hamster Ovary cells and modified by sequential deglycosylation of its carbohydrate side chains to expose alpha-mannosyl residues that mediate uptake of the therapeutic enzyme by surface mannose receptor expressed on target cells. Consequently, there is an industrial benefit to produce a recombinant β-glucocerebrosidase having naturally N-glycans with mannose-terminated structures.

Human β-glucocerebrosidase is expressed, targeted and stored into the plastidial stroma of P. tricornutum by means of the present invention. A plasmid containing a 55 amino acids bipartite topogenic signal sequence of the ATPase gamma subunit (atpC) from P. tricornutum fused in-frame with a 497 amino acids sequence coding for the mature human GBA lacking its native 39 amino acids signal sequence (SEQ ID No30) is used for the genetic transformation. The GBA protein contains 5 potential N-glycosylation sites.

a) Standard Culture Conditions of Phaeodactylum Tricornutum

Phaeodactylum tricornutum strain used to express GBA is grown and prepared for genetic transformation as in example 1.a).

b) Expression Constructs for Genetic Transformation

The vector used for the expression of human GBA is the same vector used for the expression of the chimeric protein EPO-eGFP in example 1.b).

The sequence containing the bipartite topogenic signal sequence fused in-frame with the human GBA (nucleic acid sequence SEQ ID No30) is synthesized with the addition of EcoRI and HindIII restriction sites flanking the 5′ and 3′ ends respectively. Alternatively, a similar sequence containing a histidine tag at the carboxy-terminal (GBA-HisTag) is also synthesized (nucleic acid sequence SEQ ID No31). After digestion by EcoRI and HindIII, each insert is introduced into the pPHA-T1 vector. As a control, an empty pPha-T1 vector lacking the GBA coding sequence is used.

c) Genetic Transformation

The genetic transformation carried out in this experiment is described in the previous example 1.c).

d) Microalgae DNA Extraction

The DNA extraction carried out in this experiment is described in the previous example 1.d).

e) Polymerase Chain Reaction (PCR) Analysis

The incorporation of the heterologous human GBA sequence in the genome of Phaeodactylum tricornutum is assessed by PCR analysis. The sequences of primers used for the PCR amplification are 5′-ATACCAAGCTCAAGATACC-3′ (SEQ ID No32) and 5′-AACTGTAACTTGTGCTCAGC-3′ (SEQ ID No33) located in the GBA coding sequence. The PCR reaction and agarose electrophoresis of PCR products are carried out as in example 1.e).

f) Immunoblotting Analysis

Intracellular fractions of wild-type and transformed cells of P. tricornutum are prepared as previously described in example 1.g).

Ten μL of intracellular fractions from the various GBA expressing cells and wild-type cells are separated by SDS-PAGE using a 12% polyacrylamide gel. The separated proteins are transferred onto nitrocellulose membrane and stained with Ponceau Red in order to control transfer efficiency. Immunoblotting experiment is performed as described in example 1.g) except that the primary antibody is an anti-GBA (Santa Cruz, sc-100544) (1:1000 in TBS-T containing milk 1% for 2 h at room temperature) and the secondary antibody is a horseradish peroxidase-conjugated bovine anti-mouse IgG (Santa Cruz, SC2371) (1:10000 in TBS-T containing milk 1% for 1.5 h at room temperature).

Deglycosylation assay is performed on the various intracellular fractions as described previously in example 1.g) and analysed by immunoblotting experiment.

g) Purification of the β-glucocerebrosidase

β-glucocerebrosidase carrying the histidine tag (GBA-HisTag) is purified from intracellular fractions by chromatography method as described in example 1.h). Purified β-glucocerebrosidase is then analysed by immunoblotting experiment.

h) Structural characterization of N-linked glycans of the β-glucocerebrosidase

N-linked glycans are released from the β-glucocerebrosidase purified by affinity chromatography and analyzed by mass spectrometry as previously described in example 1.i).

### Example 3

### Targeting of Nuclear-Encoded Viral Envelope Glycoprotein into the Plastid of Phaeodactylum tricornutum

The envelope spike of HIV contains various highly glycosylated proteins including gp120. Native N-linked glycans of gp120 are almost entirely oligomannose (Man₅₋₉GlcNAc₂) compared to the recombinant gp120 produced in the human cell line HEK293T which contains a majority of complex glycans. High-mannose glycans of gp120 (Man₆₋₉GlcNAc₂) are important determinant of antibodies recognition including 2G12, one of the most effective HIV neutralizing antibody. In the context of the viral vaccination design, the present invention thus confers a major advantage for the production of the envelope glycoprotein gp120 bearing high-mannose glycans, and used as antigens.

The viral envelope glycoprotein gp120 was expressed, targeted and stored into the plastidial stroma of P. tricornutum by means of the present invention. A plasmid containing a 55 amino acids bipartite topogenic signal sequence of the ATPase gamma subunit (atpC) from P. tricornutum fused in-frame with a 479 amino acids sequence coding for gp120 (SEQ ID No34) was used for the genetic transformation. The envelope glycoprotein gp120 contained 24 putative N-glycosylation sites.

a) Standard Culture Conditions of Phaeodactylum Tricornutum

Phaeodactylum tricornutum strain used to express gp120 was grown and prepared for genetic transformation as in example 1.a).

b) Expression Constructs for Genetic Transformation

The vector used for the expression of gp120 was the same vector used for the expression of the chimeric protein EPO-eGFP in example 1.b).

Sequences containing the bipartite topogenic signal sequence fused in-frame with gp120 with or without the addition of the eGFP coding sequence were synthesized with EcoRI and HindIII restriction sites flanking the 5′ and 3′ ends respectively (gp120-eGFP) (nucleic acid sequence SEQ ID No34 and SEQ ID No45). Alternatively, a sequence containing an histidine tag fused at the carboxy-terminal end of gp120 (gp120-HisTag) (nucleic acid sequence SEQ ID No35) was also synthesized with the addition of EcoRI and HindIII restriction sites flanking the 5′ and 3′ ends respectively. After digestion by EcoRI and HindIII, each insert was introduced into the pPHA-T1 vector. As a control, an empty pPha-T1 vector lacking the gp120 coding sequence was used.

c) Genetic Transformation

The genetic transformation carried out in this experiment is described in the previous example 1.c).

d) Microalgae DNA Extraction

The DNA extraction carried out in this experiment is described in the previous example 1.d).

e) Polymerase Chain Reaction (PCR) Analysis

The incorporation of the heterologous viral gp120 sequence in the genome of Phaeodactylum tricornutum was assessed by PCR analysis. The sequences of primers used for the PCR amplification are 5′-CACCTCAGTCATTACACAGGC-3′ (SEQ ID No36) and 5′-CCTCCTGAGGATTGCTTAA-3′ (SEQ ID No37) located in the gp120 coding sequence. The PCR reaction and agarose electrophoresis of PCR products were carried out as in example 1.e).

Results revealed a single band at 510 bp for cells transformed with the various constructs containing gp120 coding sequence (data not shown). No band was detected in cells transformed with the control vector. This result validated the incorporation of the exogenous viral gene in the genome of Phaeodactylum tricornutum.

f) Immunoblotting Analysis

Intracellular fractions of wild-type and transformed cells of P. tricornutum were prepared as previously described in example 1.g).

Ten μL of intracellular fractions from the various gp120 expressing cells and wild-type cells were separated by SDS-PAGE using a 12% polyacrylamide gel. The separated proteins were transferred onto nitrocellulose membrane and stained with Ponceau Red in order to control transfer efficiency. Immunoblotting experiment was performed as described in example 1.g) with an horseradish peroxidase-conjugated anti-eGFP (Santa Cruz, sc-9996-HRP) (1:2000 in TBS-T containing milk 1% for 2 h at room temperature).

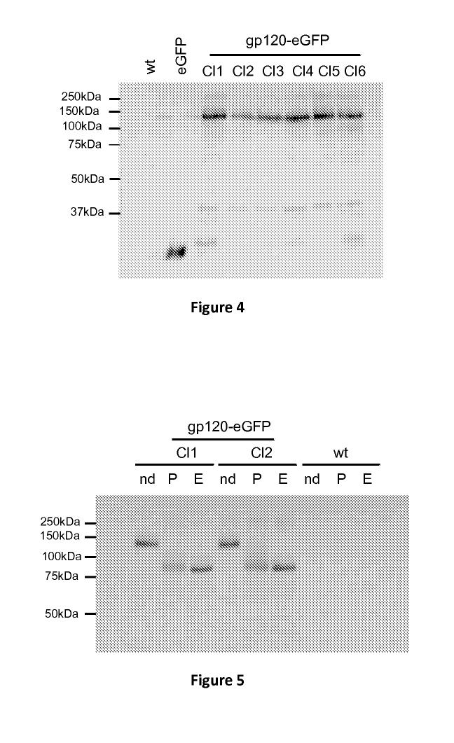

Samples from 6 transformed cell lines expressing gp120-eGFP fused to the bipartite topogenic signal sequence, a wild-type cell line and eGFP (produced in E. coli) were run on a polyacrilamide gel in order to detect gp120-eGFP by western blot. As depicted in FIG. 4, no band was visible in the sample from the wild-type cell line. Detection with anti-eGFP antibody showed a major band around 130 kDa in gp120-eGFP transformed samples. Predicted molecular weight of the corresponding amino acids sequence is around 85 kDa after the signal peptide is being cleaved. As murine gp120 contains 24 putative N-glycosylation sites, the 130 kDa band suggested heavy glycosylation of the plastid-targeted gp120-eGFP.

To further characterized glycans attached to plastid-targeted gp120-eGFP, deglycosylation assays were performed as described in example 1.g). As depicted in FIG. 5, samples from 2 cell lines expressing plastid-targeted gp120-eGFP were both deglycosylated by PNGase F and endoglycosidase H. Bands with similar apparent size of 81 kDa were observed for both treatments in accordance with the predicted molecular weight of the amino acid backbone. This result revealed that plastid targeted gp120-eGFP was fully-deglycosylated by either PNGase F or endoglycosidase H thereby indicating that N-glycans were oligomannose. The apparent shift of 50 kDa suggested the occupancy of a large number of the 24 putative N-glycosylation sites by high-mannose glycans. Indeed, Man₉GlcNAc₂ oligosaccharides attached to all putative N-glycosylation sites would give an estimated mass of 45 kDa as determined by GlycanMass analysis tool (accessible on line at http://web.expasy.org/glycanmass).

g) Purification of the Glycoprotein gp120

The glycoprotein gp120 carrying the histidine tag is purified from intracellular fractions by chromatography method as described in example 1.h). Purified gp120 is then analysed by immunoblotting experiment.

h) Structural Characterization of N-Linked Glycans of gp120

N-linked glycans are released from gp120 purified by affinity chromatography and analyzed by mass spectrometry as previously described in example 1.i).

1-13. (canceled)

14. A transformed microalga producing at least one protein harboring a “high mannose” pattern of glycosylation in the plastid of said transformed microalga, wherein
1) said transformed microalga has a Chloroplast Endoplasmic Reticulum (CER);
2) said microalga has been transformed with a nucleic acid sequence operatively linked to a promoter, said nucleic acid sequence encoding an amino acid sequence comprising:
(i) An amino-terminal bipartite topogenic signal (BTS) sequence composed of at least a signal peptide followed by a transit peptide; and
(ii) The sequence of said protein;
3) the xylosyltransferases and fucosyltransferases of said microalga have not been inactivated;
4) The N-acetylglycosyltransferase I of said microalga has not been inactivated, preferably the N-acetylglycosyltranferases II, III, IV, V and VI, mannosidase II and glycosyltransferases of said microalga have not been inactivated.

15. The transformed microalga of claim 14, wherein said protein harboring a “high mannose” pattern of glycosylation in the plastid of said microalga presents a homogenous pattern of glycosylation with at least 70% “high mannose” N-glycans, and preferably does not comprise galactose, sialic acid, fucose and/or xylose on N-glycans.

16. The transformed microalga of claim 14 wherein said microalga having a CER is selected from the group comprising heterokonts, cryptophytes and haptophytes microalgae, preferably from the group comprising Phaeodactylum, Nannochloropsis, Nitzschia, Skeletonema, Chaetoceros, Odontella, Amphiprora, Thalassiosira, Emiliania, Pavlova, Isochrysis, Apistonema and Rhodomonas, and most preferably said microalga is the diatom Phaeodactylum tricornutum.

17. The transformed microalga of claim 14, wherein the bipartite topogenic signal sequence (BTS), in this transformed microalga having a CER, enables the expression and glycosylation of said protein in the Endoplasmic Reticulum followed by a transport into the plastid of said microalga without any passage through the Golgi apparatus.

18. The transformed microalga of claim 14, wherein said protein is a heterologous protein.

19. The transformed microalga of claim 14, wherein said protein present a pattern of glycosylation with at least one exposed mannose residue and between five to nine mannose residues, preferably from six to nine mannose residues, on the oligosaccharides located at the level of the asparagine residues of the consensus sequences Asn-X-Ser/Thr, when X is different than proline and aspartic acid, of said protein.

20. The transformed microalga of claim 14, wherein said protein is selected in the group comprising lysosomal enzymes, viral envelope glycoproteins, antibodies or antibodies' fragments and derivatives thereof.

21. The transformed microalga of claim 14, wherein said amino acid sequence encoding said protein is selected from the group comprising the amino acid sequences as listed in the following table and derivatives thereof:

| | CDS SEQ | Accession | |
|---|---|---|---|
| PROTEIN | ID N° | number (Protein) | Comments |
| β-glucocerebrosidase = | SEQ ID N° 7 | AAA35873 | Lysosomal enzyme |
| Acid β-glucosidase | | | |
| α-Galactosidase A | SEQ ID N° 8 | NP_000160 | Lysosomal enzyme |
| Alglucosidase = | SEQ ID N° 9 | NP_000143 | Lysosomal enzyme |
| Acid α-glucosidase | | | |
| α-L-iduronidase | SEQ ID N° 10 | NP_000194 | Lysosomal enzyme |
| Iduronate 2-sulfatase | SEQ ID N° 11 | NP_000193 | Lysosomal enzyme |
| Arylsulfatase B | SEQ ID N° 12 | NP_000037 | Lysosomal enzyme |
| Acid Sphingomyelinase | SEQ ID N° 13 | NP_000534 | Lysosomal enzyme |
| Lysosomal acid lipase | SEQ ID N° 14 | NP_001121077 | Lysosomal enzyme |
| GP120 | SEQ ID N° 15 | NP_579894 | Envelope |
| | | | glycoprotein |
| | | | from Human |
| | | | Immunodeficiency |
| | | | Virus 1 |
| GP41 | SEQ ID N° 16 | NP_579895 | Envelope |
| | | | transmembrane |
| | | | glycoprotein |
| | | | from Human |
| | | | Immunodeficiency |
| | | | Virus 1 |
| E1 protein | SEQ ID N° 17 | From aa 192 to 383 | Envelope |
| | | of the polyprotein | glycoprotein from |
| | | P27958 | Hepatitis C Virus |
| E2 protein | SEQ ID N° 18 | From aa 384 to 746 | Envelope |
| | | of the polyprotein | glycoprotein from |
| | | P27958 | Hepatitis C Virus |
| E protein | SEQ ID N° 19 | From aa 281 to 775 | Envelope |
| | | of the polyprotein | glycoprotein from |
| | | ADO97105 | Dengue virus 1 |
| E protein | SEQ ID N° 20 | From aa 291 to 791 | Envelope |
| | | of the polyprotein | glycoprotein from |
| | | ADL27981 | West Nile Virus |
| Spike glycoprotein | SEQ ID N° 21 | ACI28632 | Envelope |
| precursor | | | glycoprotein from |
| | | | Ebola virus |
| immunoglobulin | SEQ ID N° 22 | CAC20454 | Gamma 1 |
| heavy chain | SEQ ID N° 23 | CAC20457 | Gamma 4 |
| constant region gamma | | | |
| Immunoglobulin | SEQ ID N° 24 | AAA59127 | |
| Variable Heavy Chain | | | |
| Immunoglobulin Kappa | SEQ ID N° 25 | CAA09181 | |
| light Chain (VL + CL) | | | |

22. A method for producing at least one protein harboring a “high mannose” pattern of glycosylation in the plastid of a transformed microalga producing at least one protein harboring a “high mannose” pattern of glycosylation in the plastid of said transformed microalga, wherein
1) said transformed microalga has a Chloroplast Endoplasmic Reticulum (CER);
2) said microalga has been transformed with a nucleic acid sequence operatively linked to a promoter, said nucleic acid sequence encoding an amino acid sequence comprising:
(i) An amino-terminal bipartite topogenic signal (BTS) sequence composed of at least a signal peptide followed by a transit peptide; and
(ii) The sequence of said protein;
3) the xylosyltransferases and fucosyltransferases of said microalga have not been inactivated;
4) the N-acetylglycosyltransferase I of said microalga has not been inactivated, preferably the N-acetylglycosyltranferases II, III, IV, V and VI, mannosidase II and glycosyltransferases of said microalga have not been inactivated;
wherein said method comprises the steps of:
1) culturing said transformed microalga;
2) harvesting the plastid of said transformed microalga; and
3) purifying said protein from said plastid.

23. The method of claim 22 wherein said method comprises a step 4) of determining the glycosylation pattern of said protein and conserving the protein harboring a high mannose pattern of glycosylation.

24. A protein harboring a high mannose pattern of glycosylation produced by the method of claim 22.

25. A composition comprising the protein of claim 24.


SEQUENCE LISTING (45 sequences)

SEQ ID NO: 1 - protein, 46 aa, Guillardia theta
MIRACALLGLAASAAAFAPSSLPIRANRASAVSKMSMQSNRFSYRS

SEQ ID NO: 2 - protein, 55 aa, Phaeodactylum tricornutum
MRSFCIAALLAVASAFTTQPTSFTVKTANVGERASGVFPEQSSAHRTRKATIVMD

SEQ ID NO: 3 - protein, 54 aa, Phaeodactylum tricornutum
MKVATTLTLAFICCASAFGLNGQTTSVMKKVGFDAGSKPMVQAIDVQGNRLGSN

SEQ ID NO: 4 - protein, 30 aa, Phaeodactylum tricornutum
MKTAVIASLIAGAAAFAPAKNAARTSVATN

SEQ ID NO: 5 - protein, 104 aa, Phaeodactylum tricornutum
MGRGVIIFCVKNFAVWLLIITSAVSIQAWIPLPLSATVKARIDSTTLFFSRYKTPLYHGGNEESYGPPAPAVDSRYYTYVEAPVQSSRSRDTKQPITLSRFLSD

SEQ ID NO: 6 - protein, 43 aa, Phaeodactylum tricornutum
MKFTAACSIALAASASAFAPIPSVSRTTDLSMSLQKDLANVGK

SEQ ID NO: 7 - protein, 497 aa, Homo sapiens [sequence omitted]

SEQ ID NO: 8 - protein, 398 aa, Homo sapiens
LDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 9 - protein, 883 aa, Homo sapiens
AHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPVEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 10 - protein, 626 aa, Homo sapiens
APHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 11 - protein, 517 aa, Homo sapiens
TDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 12 - protein, 497 aa, Homo sapiens
SGAGASRPPHLVFLLADDLGWNDVGFHGSRIRTPHLDALAAGGVLLDNYYTQPLCTPSRSQLLTGRYQIRTGLQHQIIWPCQPSCVPLDEKLLPQLLKEAGYTTHMVGKWHLGMYRKECLPTRRGFDTYFGYLLGSEDYYSHERCTLIDALNVTRCALDFRDGEEVATGYKNMYSTNIFTKRAIALITNHPPEKPLFLYLALQSVHEPLQVPEEYLKPYDFIQDKNRHHYAGMVSLMDEAVGNVTAALKSSGLWNNTVFIFSTDNGGQTLAGGNNWPLRGRKWSLWEGGVRGVGFVASPLLKQKGVKNRELIHISDWLPTLVKLARGHTNGTKPLDGFDVWKTISEGSPSPRIELLHNIDPNFVDSSPCPRNSMAPAKDDSSLPEYSAFNTSVHAAIRHGNWKLLTGYPGCGYWFPPPSQYNVSEIPSSDPPTKTLWLFDIDRDPEERHDLSREYPHIVTKLLSRLQFYHKHSVPVYFPAQDPRCDPKATGVWGPWM

SEQ ID NO: 13 - protein, 583 aa, Homo sapiens
LSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 14 - protein, 378 aa, Homo sapiens
SGGKLTAVDPETNMNVSEIISYWGFPSEEYLVETEDGYILCLNRIPHGRKNHSDKGPKPVVFLQHGLLADSSNWVTNLANSSLGFILADAGFDVWMGNSRGNTWSRKHKTLSVSQDEFWAFSYDEMAKYDLPASINFILNKTGQEQVYYVGHSQGTTIGFIAFSQIPELAKRIKMFFALGPVASVAFCTSPMAKLGRLPDHLIKDLFGDKEFLPQSAFLKWLGTHVCTHVILKELCGNLCFLLCGFNERNLNMSRVDVYTTHSPAGTSVQNMLHWSQAVKFQKFQAFDWGSSAKNYFHYNQSYPPTYNVKDMLVPTAVWSGGHDWLADVYDVNILLTQITNLVFHESIPEWEHLDFIWGLDAPWRLYNKIINLMRKYQ

SEQ ID NO: 15 - protein, 483 aa, Human immunodeficiency virus 1
SATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 16 - protein, 345 aa, Human immunodeficiency virus 1
AVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 17 - protein, 192 aa, Hepatitis C virus
YQVRNSSGLYHVTNDCPNSSVVYEAADAILHTPGCVPCVREGNASRCWVAVTPTVATRDGKLPTTQLRRHIDLLVGSATLCSALYVGDLCGSVFLVGQLFTFSPRHHWTTQDCNCSIYPGHITGHRMAWNMMMNWSPTAALVVAQLLRIPQAIMDMIAGAHWGVLAGIKYFSMVGNWAKVLVVLLLFAGVDA

SEQ ID NO: 18 - protein, 363 aa, Hepatitis C virus
ETHVTGGNAGRTTAGLVGLLTPGAKQNIQLINTNGSWHINSTALNCNESLNTGWLAGLFYQHKFNSSGCPERLASCRRLTDFAQGWGPISYANGSGLDERPYCWHYPPRPCGIVPAKSVCGPVYCFTPSPVVVGTTDRSGAPTYSWGANDTDVFVLNNTRPPLGNWFGCTWMNSTGFTKVCGAPPCVIGGVGNNTLLCPTDCFRKYPEATYSRCGSGPRITPRCMVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEA

SEQ ID NO: 19 - protein, 495 aa, Dengue virus 1
MRCVGIGNRDFVEGLSGATWVDVVLEHGSCVTTMAKNKPTLDIELLKTEVTNPAILRKLCIEAKISNTTTDSRCPTQGEATLVEEQDANFVCRRTFVDRGWGNGCGLFGKGSLLTCAKFKCVTKLEGKIVQYENLKYSVIVTVHTGDQHQVGNETTEHGTIATITPQAPTSEIQLTDYGALTLDCSPRTGLDFNEMVLLTMKEKSWLVHKQWFLDLPLPWTSGASTSQETWNRQDLLVTFKTAHAKKQEVVVLGSQEGAMHTALTGATEIQTSGTTTIFAGHLKCRLKMDKLTLKGTSYVMCTGSFKLEKEVAETQHGTVLVQVKYEGTDAPCKIPFSTQDEKGVTQNGRLITANPIVTDKEKPVNIETEPPFGESYIVVGAGEKALKLSWFKKGSSIGKMFEATARGARRMAILGDTAWDFGSIGGVFTSVGKLVHQVFGTAYGVLFSGVSWTMKIGIGILLTWLGLNSRSTSLSMTCIAVGMVTLYLGVMVQA

SEQ ID NO: 20 - protein, 501 aa, West Nile Virus
FNCLGMSNRDFLEGVSGATWVDLVLEGDSCVTIMSKDKPTIDVKMMNMEAANLAEVRSYCYLATVSDLSTKAACPTMGEAHNDKRADPAFVCRQGVVDRGWGNGCGLFGKGSIDTCAKFACSTKAIGRTILKENIKYEVAIFVHGPTTVESHGNYSTQAGATQAGRFSITPAAPSYTLKLGEYGEVTVDCEPRSGIDTNAYYVMTVGTKTFLVHREWFMDLNLPWSSAGSTVWRNRETLMEFEEPHATKQSVIALGSQEGALHQALAGAIPVEFSSNTVKLTSGHLKCRVKMEKLQLKGTTYGVCSKAFKFLGTPADTGHGTVVLELQYTGTDGPCKVPISSVASLNDLTPVGRLVTVNPFVSVATANAKVLIELEPPFGDSYIVVGRGEQQINHHWHKSGSSIGKAFTTTLKGAQRLAALGDTAWDFGSVGGVFTSVGKAVHQVFGGAFRSLFGGMSWITQGLLGALLLWMGINARDRSIALTFLAVGGVLLFLSVNVHA

SEQ ID NO: 21 - protein, 676 aa, Cote d′Ivoire ebolavirus
MGASGILQLPRERFRKTSFFVWVIILFHKVFSIPLGVVHNNTLQVSDIDKFVCRDKLSSTSQLKSVGLNLEGNGVATDVPTATKRWGFRAGVPPKVVNCEAGEWAENCYNLAIKKVDGSECLPEAPEGVRDFPRCRYVHKVSGTGPCPGGLAFHKEGAFFLYDRLASTIIYRGTTFAEGVIAFLILPKARKDFFQSPPLHEPANMTTDPSSYYHTTTINYVVDNFGTNTTEFLFQVDHLTYVQLEARFTPQFLVLLNETIYSDNRRSNTTGKLIWKINPTVDTSMGEWAFWENKKNFTKTLSSEELSFVPVPETQNQVLDTTATVSPPISAHNHAAEDHKELVSEDSTPVVQMQNIKGKDTMPTTVTGVPTTTPSPFPINARNTDHTKSFIGLEGPQEDHSTTQPAKTTSQPTNSTESTTLNPTSEPSSRGTGPSSPTVPNTTESHAELGKTTPTTLPEQHTAASAIPRAVHPDELSGPGFLTNTIRGVTNLLTGSRRKRRDVTPNTQPKCNPNLHYWTALDEGAAIGLAWIPYFGPAAEGIYTEGIMENQNGLICGLRQLANETTQALQLFLRATTELRTFSILNRKAIDFLLQRWGGTCHILGPDCCIEPQDWTKNITDKIDQIIHDFVDNNLPNQNDGSNWWTGWKQWVPAGIGITGVIIAIIALLCICKFML

SEQ ID NO: 22 - protein, 330 aa, Homo sapiens
ASFKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 327 aa, Homo sapiens
ASFKGPSVFPLVPCSRSTSESTAALGCLVKDYFPEPVTVSWNSCALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVRVLTVLHQDWLNGKEYKCKVSNKGLPSSIEKTISKAKGQPREPQVYTLPPSQEEMTKNQVSLTCLVKGFYPSDIAVEWESNGQPEDNYKTTPPVLDSDGSFFLYSRLTVDKSRWQEGNVFSCSVMHEALHNHYTQKSLSLSPGK

SEQ ID NO: 24 - protein, 97 aa, Homo sapiens
EVQLLESGGGLVQPGGSLRLSCAASGFTFSSYAMSWVRQSPGKGLQWVSAISGSGISTYYADSVRGRFTISRDNSKNTLYLQMSSLSRGHGRILLCE

SEQ ID NO: 25 - protein, 214 aa, Homo sapiens
VIWMTQSPSLLSASTGDRVTISCRMSQGISNYLAWYQQKPGKAPDLLIYAASTLQSGVPSRFSGSGSGTDFILTISRLQSEDFAIYYCQQYYSFPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 1404 bp, artificial sequence
  CDS coding for a fusion protein between EPO and GFP
atgaaggtcgctaccacgctaactctcgcttttatctgctgcgcatctgcgtttgggttaaatggccaaactactagcgtcatgaagaaggtcggattcggcgccggaagcaagccgatggtgcaggcaatcgatgttcaaggcaaccgtcttggctccaacgctcccccacgcctcatctgcgacagtcgagttctggagaggtacatcttagaggccaaggaggcagaaaatgtcacgatgggttgtgcagaaggtcccagactgagtgaaaatattacagtcccagataccaaagtcaacttctatgcttggaaaagaatggaggtggaagaacaggccatagaagtttggcaaggcctgtccctgctctcagaagccatcctgcaggcccaggccctgctagccaattcctcccagccaccagagacccttcagcttcatatagacaaagccatcagtggtctacgtagcctcacttcactgcttcgggtactgggagctcagaaggaattgatgtcgcctccagataccaccccacctgctccactccgaacactcacagtggatactttctgcaagctcttccgggtctacgccaacttcctccgggggaaactgaagctgtacacgggagaggtctgcaggagaggggacaggctggaagttctgttccaggggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 27 - DNA, 1422 bp, artificial sequence
  CDS coding for a fusion protein EPO-GFP-HisTag
atgaaggtcgctaccacgctaactctcgcttttatctgctgcgcatctgcgtttgggttaaatggccaaactactagcgtcatgaagaaggtcggattcggcgccggaagcaagccgatggtgcaggcaatcgatgttcaaggcaaccgtcttggctccaacgctcccccacgcctcatctgcgacagtcgagttctggagaggtacatcttagaggccaaggaggcagaaaatgtcacgatgggttgtgcagaaggtcccagactgagtgaaaatattacagtcccagataccaaagtcaacttctatgcttggaaaagaatggaggtggaagaacaggccatagaagtttggcaaggcctgtccctgctctcagaagccatcctgcaggcccaggccctgctagccaattcctcccagccaccagagacccttcagcttcatatagacaaagccatcagtggtctacgtagcctcacttcactgcttcgggtactgggagctcagaaggaattgatgtcgcctccagataccaccccacctgctccactccgaacactcacagtggatactttctgcaagctcttccgggtctacgccaacttcctccgggggaaactgaagctgtacacgggagaggtctgcaggagaggggacaggctggaagttctgttccaggggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcaccaccatcaccaccattaa

SEQ ID NO: 28 - DNA, 20 bp, artificial sequence
  PCR primer
gtctatatgaagctgaaggg

SEQ ID NO: 29 - DNA, 19 bp, artificial sequence
  PCR primer
gtgagcaagggcgaggagc

SEQ ID NO: 30 - DNA, 1659 bp, artificial sequence
  CDS coding for a chimeric protein containing the bipartite topogenic signal sequence fused in-frame with the human GBA
atgagatccttttgcatcgcagccctttttgctgtggcatctgccttcaccacacagccaacttccttcactgtgaagactgcgaatgtgggcgaacgggcgagtggggttttccctgagcagagctctgctcatcgcacgcgtaaagcaacgattgtcatggatgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccagtga

SEQ ID NO: 31 - DNA, 1677 bp, artificial sequence
  CDS coding for a chimeric GBA-HisTag protein
atgagatccttttgcatcgcagccctttttgctgtggcatctgccttcaccacacagccaacttccttcactgtgaagactgcgaatgtgggcgaacgggcgagtggggttttccctgagcagagctctgctcatcgcacgcgtaaagcaacgattgtcatggatgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcatcgccagcaccaccatcaccaccattga

SEQ ID NO: 32 - DNA, 19 bp, artificial sequence
  PCR primer
ataccaagctcaagatacc

SEQ ID NO: 33 - DNA, 20 bp, artificial sequence
  PCR primer
aactgtaacttgtgctcagc

SEQ ID NO: 34 - DNA, 1605 bp, artificial sequence
  CDS coding for a chimeric protein containing the bipartite topogenic signal sequence fused in-frame with gp120 coding sequence containing a stop codon
atgagatccttttgcatcgcagccctttttgctgtggcatctgccttcaccacacagccaacttccttcactgtgaagactgcgaatgtgggcgaacgggcgagtggggttttccctgagcagagctctgctcatcgcacgcgtaaagcaacgattgtcatggataaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagatga

SEQ ID NO: 35 - DNA, 1623 bp, artificial sequence
  CDS coding for a chimeric gp120-HisTag protein
atgagatccttttgcatcgcagccctttttgctgtggcatctgccttcaccacacagccaacttccttcactgtgaagactgcgaatgtgggcgaacgggcgagtggggttttccctgagcagagctctgctcatcgcacgcgtaaagcaacgattgtcatggataaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagacaccaccatcaccaccattga

SEQ ID NO: 36 - DNA, 21 bp, artificial sequence
  PCR primer
cacctcagtcattacacaggc

SEQ ID NO: 37 - DNA, 19 bp, artificial sequence
  PCR primer
cctcctgaggattgcttaa

SEQ ID NO: 38 - DNA, 142 bp, Phaeodactylum tricornutum
gggctgcaggacgcaatggaggattatcaccgcaaaaatgaacttcgaaaaaaactttcgagcgaccatggaaaaggaggatcagattcagattacaacagtggattgctctggtagcaaatatcttctgctagattggctc

SEQ ID NO: 39 - DNA, 245 bp, Phaeodactylum tricornutum
acataccttcagcgtcgtcttcactgtcacagtcaactgacagtaatcgttgatccggagagattcaaaattcaatctgtttggacctggataagacacaagagcgacatcctgacatgaacgccgtaaacagcaaatcctggttgaacacgtatccttttgggggcctccgctacgacgctcgctccagctggggcttccttactatacacagcgcgcatatttcacggttgccagatgtcaag

SEQ ID NO: 40 - DNA, 835 bp, cauliflower mosaic virus
agattagccttttcaatttcagaaagaatgctaacccacagatggttagagaggcttacgcagcaggtctcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacg

SEQ ID NO: 41 - DNA, 240 bp, Agrobacterium tumefaciens
acgattgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatggcaattaccttattcgcaacttctttac

SEQ ID NO: 42 - DNA, 242 bp, Phaeodactylum tricornutum
accttccttaaaaatttaattttcattagttgcagtcactccgctttggtttcacagtcaggaataacactagctcgtcttcaccatggatgccaatctcgcctattcatggtgtataaaagttcaacatccaaagctagaacttttggaaagagaaagaatatccgaatagggcacggcgtgccgtattgttggagtggactagcagaaagtgaggaaggcacaggatgagttttctcgag

SEQ ID NO: 43 - DNA, 253 bp, Agrobacterium tumefaciens
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 44 - DNA, 1353 bp, artificial sequence
  BiP-eGFP-EPO-DDEL construction
atgatattcatgagaattgccgtagcagcactggccttgctggctgctccctccattcgtgccgaagaggccggtgaagaggccaagatgggtaccgtgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagctggaagttctgttccaggggcccgctcccccacgcctcatctgcgacagtcgagttctggagaggtacatcttagaggccaaggaggcagaaaatgtcacgatgggttgtgcagaaggtcccagactgagtgaaaatattacagtcccagataccaaagtcaacttctatgcttggaaaagaatggaggtggaagaacaggccatagaagtttggcaaggcctgtccctgctctcagaagccatcctgcaggcccaggccctgctagccaattcctcccagccaccagagacccttcagcttcatatagacaaagccatcagtggtctacgtagcctcacttcactgcttcgggtactgggagctcagaaggaattgatgtcgcctccagataccaccccacctgctccactccgaacactcacagtggatactttctgcaagctcttccgggtctacgccaacttcctccgggggaaactgaagctgtacacgggagaggtctgcaggagaggggacagggacgatgagttgtga

SEQ ID NO: 45 - DNA, 2346 bp, artificial sequence
  BTS-GP120-GFP construction
atgagatccttttgcatcgcagccctttttgctgtggcatctgccttcaccacacagccaacttccttcactgtgaagactgcgaatgtgggcgaacgggcgagtggggttttccctgagcagagctctgctcatcgcacgcgtaaagcaacgattgtcatggataaattgtgggtcacagtctattatggggtacctgtgtggaaggaagcaaccaccactctattttgtgcatcagatgctaaagcatatgatacagaggtacataatgtttgggccacacatgcctgtgtacccacagaccccaacccacaagaagtagtattggtaaatgtgacagaaaattttaacatgtggaaaaatgacatggtagaacagatgcatgaggatataatcagtttatgggatcaaagcctaaagccatgtgtaaaattaaccccactctgtgttagtttaaagtgcactgatttgaagaatgatactaataccaatagtagtagcgggagaatgataatggagaaaggagagataaaaaactgctctttcaatatcagcacaagcataagaggtaaggtgcagaaagaatatgcatttttttataaacttgatataataccaatagataatgatactaccagctataagttgacaagttgtaacacctcagtcattacacaggcctgtccaaaggtatcctttgagccaattcccatacattattgtgccccggctggttttgcgattctaaaatgtaataataagacgttcaatggaacaggaccatgtacaaatgtcagcacagtacaatgtacacatggaattaggccagtagtatcaactcaactgctgttaaatggcagtctagcagaagaagaggtagtaattagatctgtcaatttcacggacaatgctaaaaccataatagtacagctgaacacatctgtagaaattaattgtacaagacccaacaacaatacaagaaaaagaatccgtatccagagaggaccagggagagcatttgttacaataggaaaaataggaaatatgagacaagcacattgtaacattagtagagcaaaatggaataacactttaaaacagatagctagcaaattaagagaacaatttggaaataataaaacaataatctttaagcaatcctcaggaggggacccagaaattgtaacgcacagttttaattgtggaggggaatttttctactgtaattcaacacaactgtttaatagtacttggtttaatagtacttggagtactgaagggtcaaataacactgaaggaagtgacacaatcaccctcccatgcagaataaaacaaattataaacatgtggcagaaagtaggaaaagcaatgtatgcccctcccatcagtggacaaattagatgttcatcaaatattacagggctgctattaacaagagatggtggtaatagcaacaatgagtccgagatcttcagacctggaggaggagatatgagggacaattggagaagtgaattatataaatataaagtagtaaaaattgaaccattaggagtagcacccaccaaggcaaagagaagagtggtgcagagagaaaaaagactggaagttctgttccaggggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa